(19) United States
(12) Patent Application Publication
(10) Pub. No.: US 2018/0258418 A1
(43) Pub. Date: Sep. 13, 2018

(54) METHOD OF IDENTIFYING GENOME-WIDE OFF-TARGET SITES OF BASE EDITORS BY DETECTING SINGLE STRAND BREAKS IN GENOMIC DNA

(71) Applicant: INSTITUTE FOR BASIC SCIENCE, Daejeon (KR)

(72) Inventor: Jin-Soo KIM, Seoul (KR)

(21) Appl. No.: 15/872,907

(22) Filed: Jan. 16, 2018

Related U.S. Application Data
(60) Provisional application No. 62/446,951, filed on Jan. 17, 2017.

(51) Int. Cl. C12N 15/10; C12N 9/22; C12N 15/90; C12N 15/113

(52) U.S. Cl. CPC C12N 15/102; C12N 9/22; C12N 15/902; C12Y 305/04005; C12N 2310/11; C12N 2310/20; C12N 15/113

(57) ABSTRACT

Provided are a composition for inducing DNA single strand breaks in DNA, the composition comprising a cytidine deaminase, an inactivated target-specific endonuclease, and a guide RNA, a method for inducing a single-strand break in DNA, using the same, a method for analyzing a nucleic acid sequence of a base-editing-introduced DNA, and a method for identifying (or measuring or detecting) a base-editing site, base-editing efficiency at an on-target site, an off-target site, and/or target specificity.

### CROSS-REFERENCE TO RELATED APPLICATIONS

This application claims the benefits of U.S. Provisional Application No. 62/446,951, filed on Jan. 17, 2017, in the United States Patent and Trademark Office, the entire disclosures of which are hereby incorporated by reference.

### BACKGROUND OF THE INVENTION

### 1. Field

The present disclosure relates to a composition for inducing DNA single strand breaks in DNA, the composition comprising a cytidine deaminase, an inactivated target-specific endonuclease, and a guide RNA, a method for inducing a single-strand break in DNA, using the same, a method for analyzing a nucleic acid sequence of a base-editing-introduced DNA, and a method for identifying (or measuring or detecting) a base-editing site, base-editing efficiency at an on-target site, an off-target site, and/or target specificity.

### 2. Description of the Related Art

A base editor (programmable deaminase) comprising a DNA-binding module and a cytidine deaminase enables targeted nucleotide substitutions or base editing in a genome without producing DNA-strand breaks. Unlike programmable nucleases, such as CRISPR-Cas9 and ZFN (zinc-finger nuclease), which induce small insertions or deletions (indels) at a target site, programmable deaminases convert C to T (C to G or A, to a lesser extent) within several nucleotides at a target site. Programmable deaminases can correct point mutations causing genetic diseases or can create single-nucleotide polymorphisms (SNPs) of interest in human cells, animals, and plants.

To data, four different classes of programmable deaminases have been reported:

1) Base Editors (BEs) composed of catalytically-deficient Cas9 (dCas9) derived from S. pyogenes or D10A nickase (nCas9) and rAPOBEC1, a cytidine deaminase from rats; 2) Target-AID composed of dCas9 or nCas9 and PmCDA1, an activation-induced cytidine deaminase (AID) ortholog from sea lamprey2, or human AID; 3) CRISPR-X composed of dCas9 and sgRNAs linked to MS2 RNA hairpins to recruit a hyperactive AID variant fused to MS2-binding protein; and 4) Zinc-finger proteins or transcription activator-like effectors (TALEs) fused to a cytidine deaminase.

In spite of broad interest in base editing with a base editor, appropriate methods have not yet been developed for analyzing genome-wide target specificities of programmable deaminases. There is therefore a need for the development of a tool capable of analyzing genome-wide target specificities of base editors, thereby analyzing the base editors for base editing efficiency, off-target sites, and off-target effects.

### SUMMARY OF THE INVENTION

Provided in the present specification are a means for analyzing genome-wide target specificities of a base editor and a means for analyzing off-target sites and off-target effects of the base editor through the analysis of genome-wide target specificities. An aspect provides a composition for producing single-strand breaks in DNA, the composition comprising: (a) a deaminase or a gene coding therefor (cDNA, rDNA, or mRNA); (b) an inactivated target-specific endonuclease or a gene coding therefor (cDNA, rDNA, or mRNA); and (c) a guide RNA or a gene coding therefor. The composition may not contain a uracil-specific excision reagent (USER).

Another aspect provides a method for inducing a single-strand break in DNA, the method comprising a step of introducing into a cell or contacting with DNA separated from cells, (a) a deaminase or a gene coding therefor (cDNA, rDNA, or mRNA), (b) an inactivated target-specific endonuclease or a gene coding therefor (cDNA, rDNA, or mRNA), and (c) a guide RNA or a gene coding therefor. This method may not comprise a step of treating with a Uracil-Specific Excision Reagent (USER).

Another aspect provides a method for analyzing a nucleic acid sequence of DNA to which base editing is introduced by deaminase, the method comprising the steps of:

(i) introducing into a cell or contacting with DNA separated from cells (a) a deaminase or a gene coding therefor (cDNA, rDNA, or mRNA), (b) an inactivated target-specific endonuclease or a gene coding therefor(cDNA, rDNA, or mRNA), and (c) a guide RNA or a gene coding therefor to induce a single-strand break in the DNA; and

(ii) analyzing a nucleic acid sequence of a DNA fragment in which the single-strand break has been induced. The method may not comprise a step of treating with a uracil-specific excision reagent (USER) to produce a double-strand break in DNA.

Another aspect provides a method for identifying (or measuring or detecting) a base-editing site, a single-strand break site, base editing efficiency at an on-target site, an off-target site, and target specificity of deaminase, the method comprising the steps of:

(i) introducing to a cell or contacting with DNA separated from cells (a) a deaminase or a gene coding therefor (cDNA, rDNA, or mRNA), (b) an inactivated target-specific endonuclease or a gene coding therefor(cDNA, rDNA, or mRNA), and (c) a guide RNA or a gene coding therefor to induce a single-strand break in DNA;

(ii) analyzing a nucleic acid sequence of the cleaved DNA fragment; and

(iii) identifying a single-strand break site from the nucleic acid sequence data obtained by the analysis. The method may further comprise, for example, the step of (iii-1) identifying base editing (e.g., conversion of cytosine (C) to uracil (U) or thymine (T)) in the nucleic acid sequence data obtained by the analysis (sequence read) between steps (ii) and (iii) or concomitantly with, prior to or after step (iii). The method may not comprise a step of treating with a uracil-specific excision reagent (USER) to induce a double-strand break in DNA. In one embodiment, the method (for identifying, for example, base editing efficiency at an on-target site, and an off-target site) may further comprise, after step (iii), a step of (iv) identifying (determining) the break site as an off-target site when the break site is not within an on-target site.

### BRIEF DESCRIPTION OF THE DRAWINGS

FIG. 1 is a representative IGV image showing straight alignments of sequence reads at the EMX1 on-target site.

FIG. 2 shows the number of nicked sites at which sequence reads have uniform alignment only in one strand obtained as a result of the Digenome-seq (sites (reads) at which the 5′ ends have straight alignment) and the number of PAM-containing sites with 10 or fewer mismatches among the sites.

FIG. 3a is a cleavage map of the rAPOBEC1-XTEN-dCas9-NLS vector.

FIG. 3b is a cleavage map of the rAPOBEC1-XTEN-dCas9-UGI-NLS vector.

FIG. 3c is a cleavage map of the rAPOBEC1-XTEN-Cas9n-UGI-NLS vector.

FIG. 4 is a cleavage map of the Cas9 expression plasmid vector.

FIG. 5 is a cleavage map of the pET28b-BE1 vector.

FIG. 6 is a cleavage map of the pET28b-BE3 delta UGI vector.

FIG. 7 is a schematic diagram illustrating the procedure of Example 1.

### DETAILED DESCRIPTION

In the specification, Digenome-seq is modified to assess the specificity of a base sequence (e.g., Base Editor 3; BE3) composed of a Cas9 nickase and a deaminase in the human genome. Genomic DNA is treated with BE3 and a guide RNA in vitro to identify the production of a break in a single strand of the DNA double helix. BE3 off-target sites are then computationally identified from whole genome sequencing data by a method for inducing a single-strand break in DNA, using a deaminase and a method for analyzing a nucleic acid sequence, both provided in the present specification.

First of all, provided is a technique of producing double-strand breaks in DNA by using a deaminase which does not induce a double-strand break.

Another aspect provides a composition for inducing a single-strand break in DNA, the composition comprising (a) a deaminaseor a gene coding therefor (cDNA, rDNA, or mRNA), (b) an inactivated target-specific endonuclease or a gene coding therefor(cDNA, rDNA, or mRNA), and (c) a guide RNA or a gene coding therefor. The composition may not contain a uracil-specific excision reagent (USER).

The encoding gene used in the present specification may be used in the form of cDNA, rDNA or a recombinant vector carrying the same, or mRNA.

The deaminase may be cytidine deaminase. The term “cytidine deaminase”, as used herein, is intended to encompass all enzymes that have the activity of converting cytosine, which is a base existing in nucleotides (e.g., double-strand DNA, or RNA) to uracil (C-to-U conversion or C-to-U editing). The cytidine that the cytidine deaminase converts to uracil is present on a strand having PAM sequence in the sequence at an on-target site (on-target sequence). In an embodiment, the cytidine deaminase may be derived from mammals including primates such as humans, apes, etc., and rodents such as rats, mice, etc., but is not limited thereto. For example, the cytidine deaminase may be at least one selected from the group consisting of members of an APOBEC (“apolipoprotein B mRNA editing enzyme, catalytic polypeptide-like”) family, AID (activation-induced cytidine deaminase), and CDA (cytidine deaminase; e.g., CDA1), and specifically from, but not limited to, the following group:

APOBEC1: Homo sapiens APOBEC1 (proteins: GenBank Accession Nos. NP_001291495.1, NP_001635.2, and NP_005880.2; genes (as used herein, genes may refer to mRNA or cDNA) (genes encoding the proteins previously described are filled in the same order as in the proteins): GenBank Accession Nos. NM_001304566.1, NM_001644.4, and NM_005889.3), mouse (Mus musculus) APOBEC1 (proteins: GenBank Accession Nos. NP_001127863.1, and NP_112436.1; genes (genes encoding the proteins previously described are filled in the same order as in the proteins): GenBank Accession Nos. NM_001134391.1, and NM_031159.3);

APOBEC2: Homo sapiens APOBEC2 (protein: GenBank Accession No. NP_006780.1; gene: GenBank Accession No. NM_006789.3), mouse APOBEC2 (protein: GenBank Accession No. NP_033824.1; gene: GenBank Accession No. NM_009694.3);

APOBEC3B: Homo sapiens APOBEC3B (proteins: GenBank Accession Nos. NP_001257340.1, and NP_004891.4; genes (genes encoding the proteins previously described are filled in the same order as in the proteins): GenBank Accession Nos. NM_001270411.1, NM_004900.4), mouse (Mus musculus) APOBEC3B (protein: GenBank Accession Nos. NP_001153887.1, NP_001333970.1, and NP_084531.1; genes (genes encoding the proteins previously described are filled in the same order as in the proteins): GenBank Accession Nos. NM_001160415.1, NM_001347041.1, and NM_030255.3);

APOBEC3C: Homo sapiens APOBEC3C (protein: GenBank Accession No. NP_055323.2; gene: GenBank Accession No. NM_014508.2);

APOBEC3D (including APOBEC3E): Homo sapiens APOBEC3D (protein: GenBank Accession No. NP_689639.2; gene: GenBank Accession No. NM_152426.3);

APOBEC3F: Homo sapiens APOBEC3F (protein: GenBank Accession Nos. NP_660341.2, and NP_001006667.1; genes (genes encoding the proteins previously described are filled in the same order as in the proteins): NM_145298.5 and NM_001006666.1);

APOBEC3G: Homo sapiens APOBEC3G (protein: GenBank Accession Nos. NP_068594.1, NP_001336365.1, NP_001336366.1, and NP_001336367.1; genes (genes encoding the proteins previously described are filled in the same order as in the proteins): NM_021822.3, NM_001349436.1, NM_001349437.1, and NM_001349438.1);

APOBEC3H: Homo sapiens APOBEC3H (proteins: GenBank Accession Nos. NP_001159474.2, NP_001159475.2, NP_001159476.2, and NP_861438.3; genes (genes encoding the proteins previously described are filled in the same order as in the proteins): NM_001166002.2, NM_001166003.2, NM_001166004.2, and NM_181773.4);

APOBEC4 (including APOBEC3E): Homo sapiens APOBEC4 (protein: GenBank Accession No. NP_982279.1; gene: GenBank Accession No. NM_203454.2); mouse APOBEC4 (protein: GenBank Accession No. NP_001074666.1; gene: GenBank Accession No. NM_001081197.1);

Activation-induced cytidine deaminase (AICDA or AID): Homo sapiens AID (proteins: GenBank Accession Nos. NP_001317272.1, and NP_065712.1; genes (genes encoding the proteins previously described are filled in the same order as in the proteins): GenBank Accession Nos. NM_001330343.1, and NM_020661.3); mouse AID (protein: GenBank Accession No. NP_033775.1; gene: GenBank Accession No. NM_009645.2); and

CDA (cytidine deaminase; EC number 3.5.4.5; e.g., CDA1): GenBank Accession Nos. NP_001776.1 (gene: NM_001785.2), CAA06460.1 (gene: AJ005261.1), and NP_416648.1 (gene: NC_000913.3).

As used herein, the term “target-specific nuclease”, also called programmable nuclease, is intended to encompass all forms of endonucleases that can recognize and cleave specific sites on target genomic DNA.

For example, the target-specific nuclease may be at least one of all the nucleases that has the activity of recognizing and cleaving at specific nucleotide sequences of target genes and thus can cause insertions and/or deletions (Indels) in the target genes.

For example, the target-specific nuclease may be at least selected from the group consisting of, but not limited to, RGENE (RNA-guided engineered nuclease; e.g., Cas9, Cpf1, etc.) derived from the microorganism immune system CRISPR.

According to an embodiment, the target-specific nuclease may be at least one selected from the group consisting of endonucleases included in type I and/or type V of the CRIPR system, such as Cas protein (e.g., Cas9 protein (CRISPR (Clustered regularly interspaced short palindromic repeats) associated protein 9)), Cpf1 protein (CRISPR from Prevotella and Francisella 1), etc. In this regard, the target-specific nuclease may further comprise a target DNA-specific guide RNA for guiding to an on-target site in genomic DNA. The guide RNA may be one transcribed in vitro, for example, from an oligonucleotide duplex or a plasmid template, but is not limited thereto. The target-specific nuclease and the guide RNA may be used in the form of ribonucleic acid protein (RNP), and the ribonucleic acid protein may be used in a mixture of a target-specific nuclease or a gene coding therefor and a RNA or a gene coding therefor, or in a complex form in which a target-specific nuclease or a gene coding therefor is associated with a RNA or a gene coding therefor.

Cas9 protein is a main protein component of the CRISPR/Cas system, which can function as an activated endonuclease or nickase.

Cas9 protein or gene information thereof may be acquired from a well-known database such as the GenBank of NCBI (National Center for Biotechnology Information). For example, the Cas9 protein may be at least one selected from the group consisting of, but not limited to:

a Cas9 protein derived from Streptococcus sp., for example, Streptococcus pyogenes (e.g., SwissProt Accession number Q99ZW2(NP_269215.1) (encoding gene: SEQ ID NO: 4);

a Cas9 protein derived from Campylobacter sp., for example, Campylobacter jejuni;

a Cas9 protein derived from Streptococcus sp., for example, Streptococcus thermophiles or Streptocuccus aureus;

a Cas9 protein derived from Neisseria meningitidis;

a Cas9 protein derived from Pasteurella sp., for example, Pasteurella multocida; and

a Cas9 protein derived from Francisella sp., for example, Francisella novicida.

Cpf1 protein, which is an endonuclease of a new CRISPR system distinguished from the CRISPR/Cas system, is small in size compared to Cas9, requires no tracrRNA, and can function with a single guide RNA. In addition, Cpf1 can recognize thymidine-rich PAM (protospacer-adjacent motif) sequences and produces cohesive double-strand breaks (cohesive end).

For example, the Cpf1 protein may be an endonuclease derived from Candidatus spp., Lachnospira spp., Butyrivibrio spp., Peregrinibacteria, Acidominococcus spp., Porphyromonas spp., Prevotella spp., Francisella spp., Candidatus Methanoplasma), or Eubacterium spp. Examples of the microorganism from which the Cpf1 protien may be derived include, but are not limited to, Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, and Eubacterium eligens.

The target-specific endonuclease may be a microorganism-derived protein or an artificial or non-naturally occurring protein obtained by a recombinant or synthesis method. By way of example, the target-specific endonuclease (e.g., Cas9, Cpf1, and the like) may be a recombinant protein produced with a recombinant DNA. As used herein, the term “recombinant DNA (rDNA)” refers to a DNA molecule artificially made by genetic recombination, such as molecular cloning, to include therein heterogenous or homogenous genetic materials derived from various organisms. For instance, when a target-specific endonuclease is produced in vivo or in vitro by expressing a recombinant DNA in an appropriate organism, the recombinant DNA may have a nucleotide sequence reconstituted with codons selected from among codons encoding the protein of interest in order to be optimal for expression in the organism.

The term “inactivated target-specific endonuclease”, as used herein, refers to a target-specific endonuclease that lacks the endonuclease activity of cleaving a DNA duplex. The inactivated target-specific endonuclease may be at least one selected from among inactivated target-specific endonucleases that lack endonuclease activity, but retain nickase activity, and inactivated target-specific endonuclease that lack both endonuclease activity and nickase activity. In an embodiment, the inactivated target-specific endonuclease may retain nickase activity. In this case, when a cytosine base is converted to a uracil base, a nick is introduced into a strand on which cytosine-to-uracil conversion occurs, or an opposite strand thereto simultaneously or sequentially irrespective of order (for example, a nick is introduced at a position between third and fourth nucleotides in the direction toward the 5′ end of a PAM sequence on a strand opposite to a strand having the PAM sequence). The modification (mutation) of such target-specific endonucleases may include substitution of a catalytic aspartate residue (for Streptococcus pyogenes-derived Cas9 protein, for example, at least one selected from the group consisting of aspartic acid at position 10 (D10), glutamic acid at position 762 (E762), histidine at position 840 (H840), asparagine at position 854 (N854), asparagine at position 863 (N863), and aspartic acid at position 986) with a different amino acid, and the different amino acid may be alanine, but is not limited thereto.

As used herein, the expression “different amino acid” is intended to refer to an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all known variants thereof, exclusive of the amino acid having a wild-type protein retained at the original substitution position.

In one embodiment, when the inactivated target-specific endonuclease is a modified Cas9 protein, the Cas9 protein may be at least one selected from the group consisting of modified Cas9 that lacks endonuclease activity and retains nickase activity as a result of introducing mutation (for example, substitution with a different amino acid) to D10 or H840 of Streptococcus pyogenes-derived Cas9 protein (e.g., SwissProt Accession number Q99ZW2(NP_269215.1)), and modified Cas9 protein that lacks both endonuclease activity and nickase activity as a result of introducing mutations (for example, substitution with different mutations) to both D10 and H840 of Streptococcus pyogenes-derived Cas9 protein. In Cas9 protein, for example, the mutation at D10 may be D10A mutation (the amino acid D at position 10 in Cas9 protein is substituted with A; below, mutations introduced to Cas9 are expressed in the same manner), and the mutation at H840 may be H840A mutation. In one embodiment, the inactivated target-specific endonuclease may be a nickase (e.g., encoded by SEQ ID NO: 11) mutated from Streptococcus pyogenes (Streptococcus pyogenes)-derived Cas9 protein (SEQ ID NO: 4) by substituting D10 with A (D10A).

The cytidine deaminase and the inactivated target-specific endonuclease may be used in the form of a fusion protein in which they are fused to each other directly or via a peptide linker (for example, existing in the order of cytidine deaminase-inactivated target-specific endonuclease in the N- to C-terminus direction (i.e., inactivated target-specific endonuclease fused to the C-terminus of cytidine deaminase) or in the order of inactivated target-specific endonuclease-cytidine deaminase in the N- to C-terminus direction (i.e., cytidine deaminase fused to the C-terminus of inactivated target-specific endonuclease) (or may be contained in the composition), a mixture of a purified cytidine deaminase or mRNA coding therefor and an inactivated target-specific endonuclease or mRNA coding therefor (or may be contained in the composition), a plasmid carrying both a cytidine deaminase-encoding gene and an inactivated target-specific endonuclease-encoding gene (e.g., the two genes arranged to encode the fusion protein described above) (or may be contained in the composition), or a mixture of a cytidine deaminase expression plasmid and an inactivated target-specific endonuclease expression plasmid which carry a cytidine deaminase-encoding gene and an inactivated target-specific endonuclease-encoding gene, respectively (or may be contained in the composition). In one embodiment, the cytidine deaminase and the inactivated target-specific endonuclease may be in the form of a fusion protein in which they exist in the order of cytidine deaminase-inactivated target-specific endonuclease in the N- to C-terminus direction or in the order of inactivated target-specific endonuclease-cytidine deaminase in the N- to C-terminus direction, or a single plasmid in which a cytidine deaminase-encoding gene and an inactivated target-specific endonuclease-encoding gene are contained to encode the fusion protein.

So long as it carries the cytidine deaminase-encoding gene and/or the inactivated target-specific endonuclease-encoding gene and contains an expression system capable of expressing the gene in a host cell, any plasmid may be used. The plasmid contains elements for expressing a target gene, which include a replication origin, a promoter, an operator, and a terminator, and may further comprise an enzyme site suitable for introduction into the genome of a host cell (e.g., restriction enzyme site), a selection marker for identifying successful introduction into a host cell, a ribosome binding site (RBS) for translation into a protein, and/or a transcriptional regulatory factor. The plasmid may be one used in the art, for example, at least one selected from the group consisting of, but not limited to, pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19. The host cell may be selected from among cells to which base editing or a double-strand break is intended to introduced by the cytidine deaminase (for example, eukaryotic cells including mammal cells such as human cells) and all cells that can express the cytidine deaminase-encoding gene and/or the inactivated target-specific endonuclease-encoding gene into cytidine deaminase and inactivated target-specific endonuclease, respectively (for example, E. coli, etc.).

The guide RNA, which acts to guide a mixture or a fusion protein of the cytidine deaminase and the inactivated target-specific endonuclease to an on-target site, may be at least one selected from the group consisting of CRISPR RNA (crRNA), trans-activating crRNA (tracrRNA), and single guide RNA (sgRNA), and may be, in detail, a crRNA:tracrRNA duplex in which crRNA and tracrRNA is coupled to each other, or a single-strand guide RNA (sgRNA) in which crRNA or a part thereof is connected to tracrRNA or a part thereof via an oligonucleotide linker.

Concrete sequences of the guide RNA may be appropriately selected, depending on kinds of the target-specific endonucleases used, or origin microorganisms thereof, and are an optional matter which could easily be understood by a person skilled in the art.

When a Streptococcus pyogenes-derived Cas9 protein is used as a target-specific endonuclease, crRNA may be represented by the following General Formula 1:

(General Formula 1)
5′-(N_{cas9})ₗ-(GUUUUAGAGCUA)-(X_{cas9})ₘ-3′

wherein,

N_{cas9} is a targeting sequence, that is, a region determined according to a sequence at an on-target site in a target gene (i.e., a sequence hybridizable with a sequence of an on-target site), 1 represents a number of nucleotides included in the targeting sequence and is an integer of 17 to 23 or 18 to 22, for example, 20;

the region including 12 consecutive nucleotides (GUUUUAGAGCUA; SEQ ID NO: 1) adjacent to the 3′-terminus of the targeting sequence is essential for crRNA,

X_{cas9} is a region including m nucleotides present at the 3′-terminal site of crRNA (that is, present adjacent to the 3′-terminus of the essential region), and m may be an integer of 8 to 12, for example, 11 wherein the m nucleotides may be the same or different and are independently selected from the group consisting of A, U, C, and G

In an embodiment, the X_{cas9} may include, but is not limited to, UGCUGUUUUG (SEQ ID NO: 2).

In addition, the tracrRNA may be represented by the following General Formula 2:

(General Formula 2)
5′-(Y_{cas9})ₚ-
(UAGCAAGUUAAAAUAAGGCUAGUCCGUUAUCAACUUGAAAAAGUGGCAC
CGAGUCGGUGC)-3

wherein,

the region represented by 60 nucleotides (UAGCAAGUUAAAAUAAGGCUAGUCCGUUAUCAACUUGAAAAAGUGGCAC CGAGUCGGUGC; SEQ ID NO: 3) is essential for tracrRNA,

Y_{cas9} is a region including p nucleotides present adjacent to the 3′-terminus of the essential region, and p may be an integer of 6 to 20, for example, 8 to 19 wherein the p nucleotides may be the same or different and are independently selected from the group consisting of A, U, C, and G.

Further, sgRNA may form a hairpin structure (stem-loop structure) in which a crRNA moiety including the targeting sequence and the essential region thereof and a tracrRNA moiety including the essential region (60 nucleotides) thereof are connected to each other via an oligonucleotide linker (responsible for the loop structure). In greater detail, the sgRNA may have a hairpin structure in which a crRNA moiety including the targeting sequence and essential region thereof is coupled with the tracrRNA moiety including the essential region thereof to form a double-strand RNA molecule with connection between the 3′ end of the crRNA moiety and the 5′ end of the tracrRNA moiety via an oligonucleotide linker.

In one embodiment, sgRNA may be represented by the following General Formula 3:

(General Formula 3)
5′-(N_{cas9})ₗ-(GUUUUAGAGCUA)-(oligonucleotide linker)-
(UAGCAAGUUAAAAUAAGGCUAGUCCGUUAUCAACUUGAAAAAGUGGCAC
CGAGUCGGUGC)-3

wherein, (N_{cas9})₁ is a targeting sequence defined as in General Formula 1.

The oligonucleotide linker included in the sgRNA may be 3-5 nucleotides long, for example 4 nucleotides long in which the nucleotides may be the same or different and are independently selected from the group consisting of A, U, C, and G.

The crRNA or sgRNA may further contain 1 to 3 guanines (G) at the 5′ end thereof (that is, the 5′ end of the targeting sequence of crRNA).

The tracrRNA or sgRNA may further comprise a terminator inclusive of 5 to 7 uracil (U) residues at the 3′ end of the essential region (60 nt long) of tracrRNA.

The target sequence for the guide RNA may be about 17 to about 23 or about 18 to about 22, for example, 20 consecutive nucleotides adjacent to the 5′ end of PAM (Protospacer Adjacent Motif (for S. pyogenes Cas9, 5′-NGG-3′ (N is A, T, G, or C)) on a target DNA.

As used herein, the term “the targeting sequence” of guide RNA hybridizable with the target sequence for the guide RNA refers to a nucleotide sequence having a sequence complementarity of 50% or higher, 60% or higher, 70% or higher, 80% or higher, 90% or higher, 95% or higher, 99% or higher, or 100% to a nucleotide sequence of a complementary strand to a DNA strand on which the target sequence exists (i.e., a DNA strand having a PAM sequence (5′-NGG-3′ (N is A, T, G, or C))) and thus can complimentarily couple with a nucleotide sequence of the complementary strand.

In the present specification, a nucleic acid sequence at an on-target site is represented by that of the strand on which a PAM sequence exists among two DNA strands in a region of a target gene. In this regard, the DNA strand to which the guide RNA couples is complementary to a strand on which a PAM sequence exists. Hence, the targeting sequence included in the guide RNA has the same nucleic acid sequence as a sequence at an on-target site, with the exception that U is employed instead of T due to the RNA property. In other words, a targeting sequence of guide RNA and a sequence at the on-target site (or a sequence of a cleavage site) are represented by the same nucleic acid sequence with the exception that T and U are interchanged, in the present specification.

The guide RNA may be used in the form of RNA (or may be contained in the composition) or in the form of a plasmid carrying a DNA coding for the RNA (or may be contained in the composition).

The composition and method described in the present specification may comprise or may not use a Uracil-Specific Excision Reagent (USER). The term “uracil-specific excision reagent”, as used herein, is intended to encompass any material that plays a role in excising uracil residues converted from cytosine residues by the cytidine deaminase and/or inducing DNA cleavage at the uracil-excised positions.

According to an embodiment, the uracil-specific excision reagent (USER) includes uracil DNA glycosylase (UDG), endonuclease VIII, or a combination thereof. In one embodiment, the uracil-specific excision reagent may comprise endonuclease VIII or a combination of uracil DNA glycosylase and endonuclease VIII.

Uracil DNA glycosylase (UDG) is an enzyme that functions to prevent mutagenesis eliminating uracil from DNA molecules and may be at least one selected from all enzymes that play a role in cleaving the N-glycosylic bond to initiate the base-excision repair (BER) pathway. By way of example, the uracil DNA glycosylase may be at least one selected from the group consisting of, but not limited to, Escherichia coli uracil DNA glycosylases (e.g., GenBank Accession Nos. ADX49788.1, ACT28166.1, EFN36865.1, BAA10923.1, ACA76764.1, ACX38762.1, EFU59768.1, EFU53885.1, EFJ57281.1, EFU47398.1, EFK71412.1, EFJ92376.1, EFJ79936.1, EF059084.1, EFK47562.1, KXH01728.1, ESE25979.1, ESD99489.1, ESD73882.1, ESD69341.1, etc.), human uracil DNA glycosylases (e.g., GenBank Accession Nos. NP_003353.1, NP_550433.1, etc.), and mouse uracil DNA glycosylases (e.g., GenBank Accession Nos. NP_001035781.1, NP_035807.2, etc.).

Endonuclease VIII acts to excise damaged uracil residues from double-stranded DNA while eliminating the uracil-excised nucleotides and may be at least one selected from among all enzymes that have N-glycosylase activity of releasing the uracil residues damaged by uracil DNA glycosylase, generating an apurinic site (AP-site) and AP-lyase activity of cleaving 3′ and 5′ to the AP site. For example, the endonuclease VIII may be at least one selected from the group consisting of human endonuclease VIII (e.g., GenBank Accession Nos. BAC06476.1, NP_001339449.1, NP_001243481.1, NP_078884.2, NP_001339448.1, etc.), mouse endonuclease VIII (e.g., GenBank Accession Nos. BAC06477.1, NP_082623.1, etc.), and Escherichia coli endonuclease VIII (e.g., GenBank Accession Nos. OBZ49008.1, OBZ43214.1, OBZ42025.1, ANJ41661.1, KYL40995.1, KMV55034.1, KMV53379.1, KMV50038.1, KMV40847.1, AQW72152.1, etc.), but is not limited thereto.

Another aspect provides a method for inducing a double-strand break in DNA, the method comprising a step of introducing into a cell or contacting with DNA separated from cells, (a) a deaminaseor a gene coding therefor (cDNA, rDNA, or mRNA), (b) an inactivated target-specific endonuclease or a gene coding therefor (cDNA, rDNA, or mRNA), and (c) a guide RNA or a gene coding therefor. This method may not comprise a step of treating with a uracil-specific excision reagent (USER).

As such, the production (or introduction) of a single-strand break in DNA allows for analyzing sites of genomic DNA or on-target sites of DNA in which cytidine deaminase makes base editing (conversion from C to U) or produces (introduces) the single-strand break, and for base editing efficiency, whereby base editing efficiency in on-target sites, specificity for on-target sequences, and off-target sequences can be identified (or measured).

Another aspect provides a method for analyzing a nucleic acid sequence of DNA to which base editing is introduced by deaminase, the method comprising the steps of:

(i) introducing into a cell or contacting with DNA separated from cells (a) a deaminaseor a gene coding therefor (cDNA, rDNA, or mRNA), (b) an inactivated target-specific endonuclease or a gene coding therefor(cDNA, rDNA, or mRNA), and (c) a guide RNA or a gene coding therefor to induce a single-strand break in the DNA; and

(ii) analyzing a nucleic acid sequence of a DNA fragment in which the single-strand break has been induced. The method may not comprise a step of treating with a uracil-specific excision reagent (USER) to produce a double-strand break in DNA.

Another aspect provides a method for identifying (or measuring or detecting) a base-editing site, a single-strand break site, base editing efficiency at an on-target site, an off-target site, and target specificity of deaminase, the method comprising the steps of:

(i) introducing to a cell or contacting with DNA separated from cells (a) a deaminaseor a gene coding therefor (cDNA, rDNA, or mRNA), (b) an inactivated target-specific endonuclease or a gene coding therefor(cDNA, rDNA, or mRNA), and (c) a guide RNA or a gene coding therefor to induce a single-strand break in DNA;

(ii) analyzing a nucleic acid sequence of the cleaved DNA fragment; and

(iii) identifying a single-strand break site from the nucleic acid sequence data obtained by the analysis. The method may further comprise, for example, the step of (iii-1) identifying base editing (e.g., conversion of cytosine (C) to uracil (U) or thymine (T)) in the nucleic acid sequence data obtained by the analysis (sequence read) between steps (ii) and (iii) or concomitantly with, prior to or after step (iii). The method may not comprise a step of treating with a uracil-specific excision reagent (USER) to induce a double-strand break in DNA.

In one embodiment, the method (for identifying, for example, base editing efficiency at an on-target site, and an off-target site) may further comprise, after step (iii), a step of (iv) identifying (determining) the break position as an off-target site when the break position is not within an on-target site.

The deaminase, the inactivated target-specific endonuclease, the guide RNA, and the uracil-specific excision reagent are as defined above.

The methods provided in the present specification may be conducted in cells (which may be separated from a living body) or in vitro (extracellularly). For example, the methods may be executed in vitro (extracellularly). In greater detail, all the steps of the methods may be conducted in vitro. Alternatively, step (i) may be conducted in cells while step (ii) and subsequent steps may be conducted in vitro (extracellularly) with the DNA (e.g., genomic DNA) extracted from the cells in which step (i) has been conducted.

In step (i), a deaminase (or a gene coding therefor), an inactivated target-specific endonuclease (or a gene coding therefor), and a guide RNA are transfected into cells or are contacted (e.g., incubated) with DNA extracted from cells to induce base editing (base conversion, e.g., from cytosine to uracil) at an on-target site targeted by the guide RNA and the generation of nicks in a single strand of the DNA. The cells may be selected from among all eukaryotic cells to which base editing and/or single-strand breaks by deaminase are to be introduced, and from among, for example, mammal cells including human cells.

The transfection may be performed using any typical method for introducing to cells

(1) a mixture of a deaminase, an inactivated target-specific endonuclease, and a guide RNA or a complex in which they are associated with one another (ribonucleic acid protein; RNP),

(2) a mixture of a deaminase-encoding mRNA, an inactivated target-specific endonuclease-encoding mRNA, and a guide RNA,

(3) a plasmid (recombinant vector) carrying both a deaminase-encoding gene and a target-specific endonuclease-encoding gene or plasmids (recombinant vectors) respectively carrying a deaminase-encoding gene and a target-specific endonuclease-encoding gene, and a guide RNA or a plasmid carrying a guide RNA-encoding gene. By way of example, the introduction may be conducted by electroporation, lipofection, microinjection, etc., but is not limited thereto.

In one embodiment, the step (i) may be carried out by incubating a DNA extracted from cells (which is to be identified for the base editing (base-editing site, base editing efficiency, etc.) and/or single-strand break (cleavage positions, cleavage efficiency, etc.) by a deaminase and an inactivated endonuclease) with a deaminase and an inactivated target-specific endonuclease (e.g., a fusion protein containing both a cytidine deaminase and an inactivated Cas9 protein), and a guide RNA (in vitro). The DNA extracted from cells may be a genome DNA, a target gene, or a PCR (polymerase chain reaction) product inclusive of the target gene.

Optionally, a step of removing the deaminase, the inactivated target-specific endonuclease, and/or the guide RNA, all used in step (i), may be further comprised after step (i) and before step (ii). In addition, the method may further comprise a step of making blunt (or repairing) an end of the double-strand DNA fragment in which a single-strand break has been generated, after step (i) and before step (ii). The step of making an end blunt may include (b) a 3′ -to-5′ trimming step in which elimination (excision) is made of the overhangs at the 3′ end of the uncleaved strand of the double-strand DNA fragment where a single-strand break has been induced and/or (c) a 5′-to-3′ DNA synthesis step in which extension is made of the 3′-terminal nucleotide from the break point of the cleaved strand of the double-strand DNA fragment where a single-strand break has been induced (see diagram in Example 1). The 3′-to-5′ trimming step may be carried out using a suitable typical exonuclease. The 5′-to-3′ DNA synthesis step may be carried out using a suitable typical DNA polymerase.

Optionally, the method may further comprise, after step (i) and before step (ii), a step of amplifying the DNA fragment in which a single-strand break has been induced (of the DNA duplex, an oligonucleotide composed of 10 to 30 or 15 to 25 consecutive nucleotides inclusive of the cleavage site of the cleaved strand and/or an oligonucleotide composed of 10 to 30 or 15 to 25 consecutive nucleotides, corresponding (complementary) to the cleavage site, of the uncleaved strand in order to facilitate the nucleic acid sequence analysis of the DNA fragment in step (ii). For use in analysis in step (ii), the DNA fragment where a single-strand break has been induced may comprise an oligonucleotide composed of 10 to 30 or 15 to 25 consecutive nucleotides inclusive of the cleavage site of the cleaved strand and/or an oligonucleotide composed of 10 to 30 or 15 to 25 consecutive nucleotides, corresponding (complementary) to the cleavage site, of the uncleaved strand; and/or an amplification product of the oligonucleotide.

Thanks to being used together with the guide RNA, the deaminase and the inactivated target-specific endonuclease show sequence specificity and, for the most part, act on target sites (on-target). Depending on the extent to which sequences similar to the target sequence exist in sites except the on-target site, however, the side effect of acting on off-target sites may occur. As used herein, the term “off-target site” refers to a site which is not the on-target site of the deaminase and the inactivated target-specific endonuclease, but allows he deaminase and the inactivated target-specific endonuclease to be active therein, that is, a site, except the on-target site, in which base editing and/or cleavage is induced by the deaminase and the inactivated target-specific endonuclease. In one embodiment, the off-target site is intended to encompass not only actual off-target sites but also potential sites which are likely to be off-target sites.

The off-target site may include, but is not limited to, all sites, except the on-target site cleaved in vitro by the deaminase and the inactivated target-specific endonuclease.

There are various causes that make the deaminase and the inactivated target-specific endonuclease be active in sites except the on-target site. For example, the deaminase and the inactivated target-specific endonuclease may be apt to work for non-target sequences (off-target sequences) which have high sequence homology to a target sequence due to a low level of nucleotide mismatch with the target sequence designed for an on-target site.

The off-target site may be a sequence site (gene region) that satisfies at least one of the following conditions:

The number of DNA reads of which the 5′ ends are vertically aligned is 2 or greater, for example, 3 or greater, 4 or greater, 5 or greater, 6 or greater, 7 or greater, 8 or greater, 9 or greater, or 10 or greater;

A strand complementary to the strand on which a break has been induced in a double-stranded DNA fragment includes a PAM sequence;

A complementary strand to the strand on which a break has been induced in a double-stranded DNA fragment includes 15 or less or 10 or less nucleotide mismatches with a sequence at the on-target site (target sequence), for example, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, or 1 nucleotide mismatches; and

A complementary strand to the strand on which a break has been induced in a double-stranded DNA fragment includes base editing (conversion of at least cytosine (C) residue to uracil (U) or thymine (T)).

The working of the deaminase and the inactivated target-specific endonuclease in an off-target site may incur undesirable mutation in a genome, which may lead to a significant problem. Hence, a process of accurately detecting and analyzing an off-site sequence may be as very important as the activity of the deaminase and the inactivated target-specific endonuclease at an on-target site. The process may be useful for developing a deaminase and an inactivated target-specific endonuclease which both work specifically only at on-target sites without the off-target effect.

Because the cytidine deaminase and the inactivated target-specific endonuclease have activities in vivo and in vitro for the purpose of the present invention, the enzymes can be used in detecting in vitro an off-target site of DNA (e.g., genomic DNA). When applied in vivo, thus, the enzymes are expected to be active in the same sites (gene loci including off-target sequences) as the detected off-target sites.

The step (ii) in which a nucleic acid sequence of the DNA fragment cleaved (single-strand breaks) in step (i) is analyzed may be carried out using any typical nucleic acid analysis method. For example, when the separate DNA used in step (i) is a genomic DNA, the nucleic acid sequence analysis may be conducted by whole genome sequencing. In contrast to the indirect method in which a sequence having a homology with the sequence at an on-target site is searched for and would be predicted to be off-target site, whole genome sequencing allows for detecting an off-target site actually cleaved by the target-specific nuclease at the level of the entire genome, thereby more accurately detecting an off-target site.

As used herein, the term “whole genome sequencing” (WGS) refers to a method of reading the genome by many multiples such as in 10×, 20×, and 40× formats for whole genome sequencing by next generation sequencing. The term “Next generation sequencing” means a technology that fragments the whole genome or targeted regions of genome in a chip-based and PCR-based paired end format and performs sequencing of the fragments by high throughput on the basis of chemical reaction (hybridization).

In the step (iii), a DNA cleavage site is identified (or determined) using the base sequence data (sequence read) obtained in step (ii). By analyzing sequencing data, an on-target site and an off-target site can simply be detected. The determination of a site at which DNA is cleaved from the base sequence data can be performed by various approaches. In the specification, various reasonable methods are provided for determining the site. However, they are merely illustrative examples that fall within the technical spirit of the present invention, but are not intended to limit the scope of the present invention.

As an example of determining a cleaved site, when the sequence reads obtained by whole genome sequencing are aligned according to sites on a genome, the site at which the 5′ ends are vertically aligned may mean the site at which DNA is cleaved. The alignment of the sequence reads according to sites on genomes may be performed using an analysis program (for example, BWA/GATK or ISAAC). As used herein, the term “vertical alignment” refers to an arrangement in which the 5′ ends of two more sequence reads start at the same site (nucleotide position) on the genome for each of the adjacent Watson strand and Crick strand when the whole genome sequencing results are analyzed with a program such as BWA/GATK or ISAA. Through this method, the DNA fragments that are cleaved in step (ii) and thus have the same 5′ end are each sequenced.

That is, when the cleavage in step (i) occurs at on-target sites and off-target sites, the alignment of the sequence reads allows the vertical alignment of the common cleaved sites because each of their sites start at the 5′ end. However, the 5′ end is not present in the uncleaved sites, so that it can be arranged in a staggered manner in alignment. Accordingly, the vertically aligned site may be regarded as a site cleaved in step (i), which means an on-target site or off-target site cleaved by the inactivated target-specific endonuclease.

The term “alignment” means mapping sequence reads to a reference genome and then aligning the bases having identical sites in genomes to fit for each site. Accordingly, so long as it can align sequence reads in the same manner as above, any computer program may be employed. The program may be one already known in the pertinent art or may be selected from among programs tailored to the purpose. In one embodiment, alignment is performed using ISAAC, but is not limited thereto. As a result of the alignment, the site at which the DNA is cleaved by the deaminase and the inactivated target-specific endonuclease can be determined by a method such as finding a site where the 5′ end is vertically aligned as described above, and the cleaved site may be determined as an off-target site if not an on-target site. In other words, a sequence is an on-target site if identical to the base sequence designed as an on-target site of the deaminase and inactivated target-specific endonuclease, and is regarded as an off-target site if not identical to the base sequence. This is obvious according to the definition of an off-target site described above.

The method (e.g., method for identifying base editing efficiency at an on-target site and determining an off-target site) may further include a step of identifying (determining) the cleavage site to be an off-target site if the cleavage site is not an on-target site, after step (iii).

The cleaved strands of DNA fragments cleaved by a base editor (deaminase and inactivated target-specific endonuclease) may have 5′ ends vertically aligned. According to the number of DNA read(s) with 5′ ends vertically aligned (as used herein, term “DNA read(s)” refers to a DNA fragment or a set of DNA fragments which have 5′ ends vertically aligned and the same nucleic acid sequence), the number of cleavage sites can be identified. For example, when the number of a DNA read is 1, cleavage by the base editor can be determined to occur only at one site, that is, the on-target site. When the number of the DNA reads of which the 5′ ends are vertically aligned is 2 or greater, for example, 3 or greater, 4 or greater, 5 or greater, 6 or greater, 7 or greater, 8 or greater, 9 or greater, or 10 or greater, cleavage occurs at two or more sites, indicating that DNA was cleaved at at least one site which is not an on-target site (off-target site). DNA reads the 5′ ends of which are vertically aligned can be identified (or determined) to be off-target sites if they are not an on-target site (that is, have nucleic acid sequences different from that of the on-target site).

Therefore, the step (iii) of identifying a site at which the single-strand is cleaved may comprise (a) identifying (or measuring) a number of DNA reads. In this regard, when the number of DNA reads the 5′ ends of which are vertically aligned are 2 or greater, for example, 3 or greater, 4 or greater, 5 or greater, 6 or greater, 7 or greater, 8 or greater, 9 or greater, or 10 or greater, DNA cleavage can be identified (or determined) to occur at one or more non-target sites (off-target sites). In this case, in addition, the step (iv) of determining an off-target site may comprise a step of (iv-1) identifying (or determining) as an off-target site at least one of two or more DNA reads of which the 5′ ends are vertically aligned if the one has a nucleic acid sequence different from that of the on-target site.

Furthermore, determining whether the off-target site includes a PAM sequence (in greater detail, whether a PAM sequence is included in a complementary strand (strand having a complementary sequence) to a DNA read of which the 5′ end is vertically aligned and which has a nucleic acid different from that of an on-target site) can exclude a site at which cleavage has been made by error, but not by the target-specific endonuclease included in the base editor, thereby further increasing accuracy for off-target sites. Thus, the step (iii) of identifying a site at which a single-strand break has been induced may further comprise a step of (b) determining whether the off-target site includes a PAM sequence, for example, whether a PAM sequence specific for the target-specific endonuclease of the base editor is included in a complementary strand (a strand having a complementary sequence) to a DNA read of cleaved DNA fragments of which the 5′ end is vertically aligned and which has a nucleic acid sequence different from that of the on-target site. In this regard, the step (iv) of identification as an off-target site may comprise a step of (iv-2) identifying (or determining), as an off-target site, a DNA read of cleaved DNA fragment of which the 5′ end is vertically aligned and which has a nucleic acid sequence different from that of the on-target site when the DNA read includes a PAM sequence specific for the target-specific endonuclease of the base editor.

In addition, the off-target site may be composed of a sequence having a homology to the sequence of an on-target site. More specifically, because a sequence at an on-target site is represented by a nucleic acid sequence on a strand including a PAM sequence, a sequence at an off-target site may be a nucleic acid sequence of a complementary strand (a strand having a complementary sequence) to a DNA read of cleaved DNA fragments of which the 5′ end is vertically aligned and which is different in nucleic acid sequence from the on-target site. In this context, the sequence at on off-target site may have one or more nucleotide mismatches with the sequence at the on-target site, more particularly, 15 or less or 10 or less, for example, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2 nucleotide mismatches.

Hence, the step (iii) of identifying a site at which a single-strand break has been induced may further comprise a step of (c) identifying (or measuring) a number of nucleotide mismatches between a complementary strand and a sequence at an on-target site, the complementary strand having a sequence complementary to a DNA read of cleaved DNA fragments of which the 5′ end is vertically aligned and which has a nucleic acid sequence different from that at the on-target site. When the number of the nucleotide mismatches is 15 or less or 10 or less, for example, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2, the occurrence of DNA cleavage at an off-target site can be identified (or determined). In this regard, the step (iv) of identifying as an off-target site may comprise a step of (iv-3) identifying (or determining° as an off-target site when a complementary strand (a strand having a complementary sequence) to a DNA read of cleaved DNA fragments of which the 5′ end is vertically aligned and which is different in nucleic acid sequence from the on-target site has 15 or fewer or 10 or fewer nucleotide mismatches with the sequence at the on-target site, for example, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2 nucleotide mismatches.

The step (iii) may include at least one of steps (a), (b), and (c) (for example, step (a) and at least one of steps (b) and (c)). When two or more of steps (a), (b), and (c) are included, they may be conducted at the same time or irrespective of the order thereof. In addition, the step (iv) may include at least of steps (iv-1), (iv-2), and (iv-3) (for example step (iv-1) and at least one of steps (iv-2) and (iv-3)). When two or more of steps (iv-1), (iv-2), and (iv-3) are included, they may be conducted at the same time or irrespective of the order thereof.

The step (iii-1) of identifying whether base editing (e.g., conversion of cytosine (C) to uracil (U) or thymine (T)) is induced may include a step of identifying (determining) whether a nucleic acid sequence of a complementary strand (a strand having a complementary sequence) to a DNA read of cleaved DNA fragments of which the 5′ end is vertically aligned and which is different in nucleic acid sequence from the on-target site contains base editing (conversion of at least one cytosine (C) residue to a uracil (U) or thymine (T) residue). In this case, the step (iv) of identifying as an off-target site [symbol] , a step of (iv-4) identifying as an off-target site when a sequence at an off-target site may be a nucleic acid sequence of a complementary strand (a strand having a complementary sequence) to a DNA read of cleaved DNA fragments of which the 5′ end is vertically aligned and which is different in nucleic acid sequence from the on-target site contains base editing (conversion of at least one cytosine (C) residue to a uracil (U) or thymine (T) residue).

In an embodiment, the step (i) is conducted with regard to the genomic DNA to induce a single-strand break and after the whole genome analysis (step (ii), the DNA reads are aligned with ISAAC to identify alignment patterns for vertical alignment at cleaved sites and staggered alignment at uncleaved sites. A unique pattern may appear at the cleavage sites as represented by a 5′ end plot.

Moreover, as a non-limiting examples, the site where two or more sequence reads corresponding to Watson strand and Crick strand are aligned vertically may be determined as an off-target site. In addition, the site where 20% or more of sequence reads are vertically aligned and the number of sequence reads having the same 5′ end in each of the Watson and Creek strands is 10 or more is determined as an off-target site position, that is, a cleavage site.

The process in steps (ii) and (iii) of the method described above may be Digenome-seq (digested-genome sequencing). For greater details, reference may be made to Korean Patent No. 10-2016-0058703 A (this document is herein incorporated by reference in its entirety).

Base editing sites and/or single-strand break sites of the deaminase, base editing efficiency at on-target sites or target specificity (i.e., [base editing or cleavage frequency at on-target sites]/[base editing or cleavage frequency over entire sequence]), and/or off-target sites (identified as base editing sites of deaminase, but not on-target sites) can be identified (or measure or detected) by the method described above.

The identification (detection) of an off-target site is performed in vitro by treating a genomic DNA with the deaminase and the inactivated target-specific endonuclease. Thus, it can be identified whether off-target effects are actually produced also in vivo in the off-target site detected by this method. However, this is merely an additional verification process, and thus is not a step that is essentially entailed by the scope of the present invention, and is merely a step that can be additionally performed according to the needs.

In the present specification, the term “off-target effect” is intended to mean a level at which base editing and/or double-strand break occurs at an off-target site. The term “indel” (insertion and/or deletion) is a generic term for a mutation in which some bases are inserted or deleted in the middle of a base sequence of DNA.

The method for inducing a single-strand break in DNA, using a cytidine deaminase and the nucleic acid sequence analysis technique using the same, both provided in the present specification, can more accurately and effectively identify base editing sites, target specificity, and/or off-target sites of the cytidine deaminase.

### EXAMPLES

Hereafter, the present invention will be described in detail by examples.

The following examples are intended merely to illustrate the invention and are not construed to restrict the invention.

### REFERENCE EXAMPLE

1. Cell Culture and Transfection

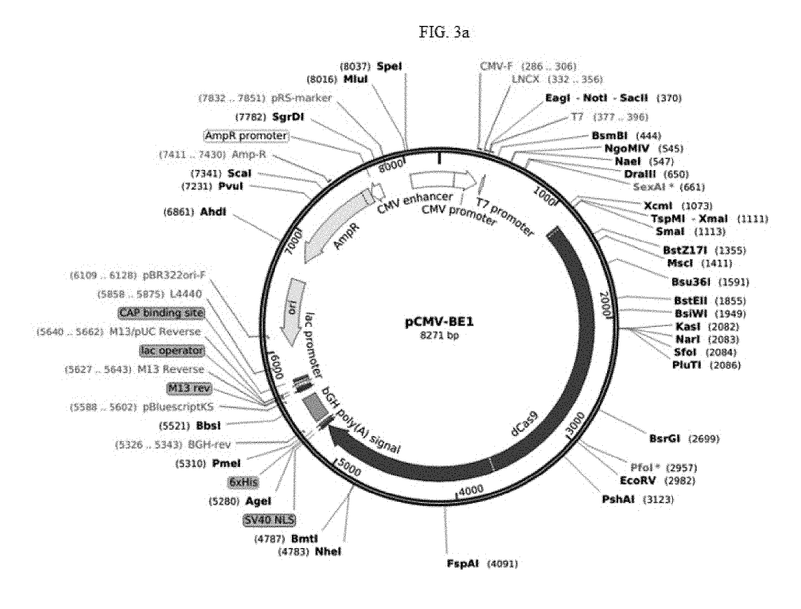

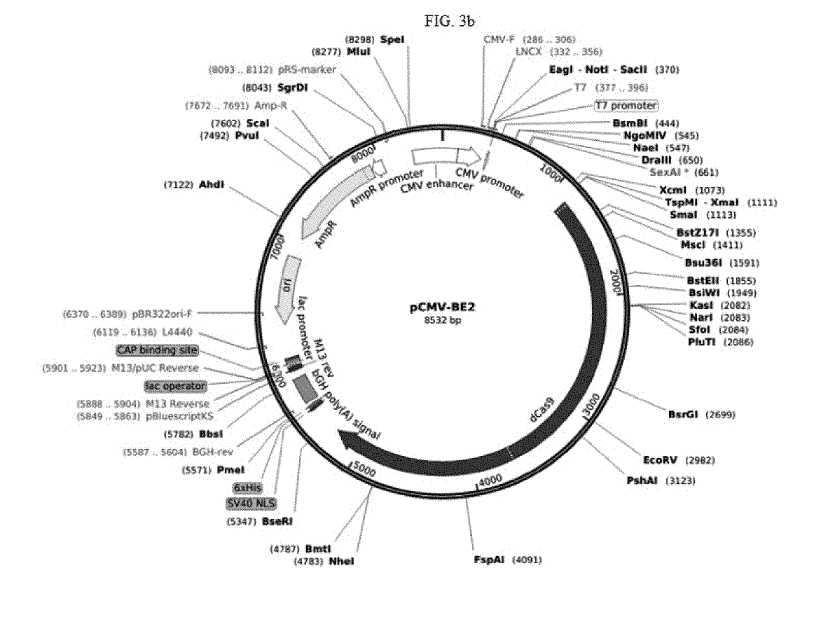

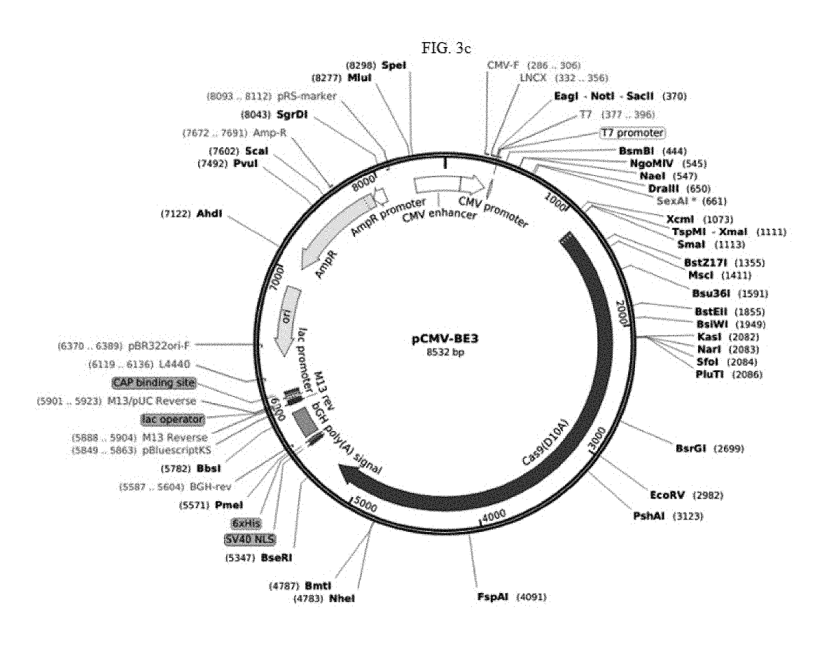

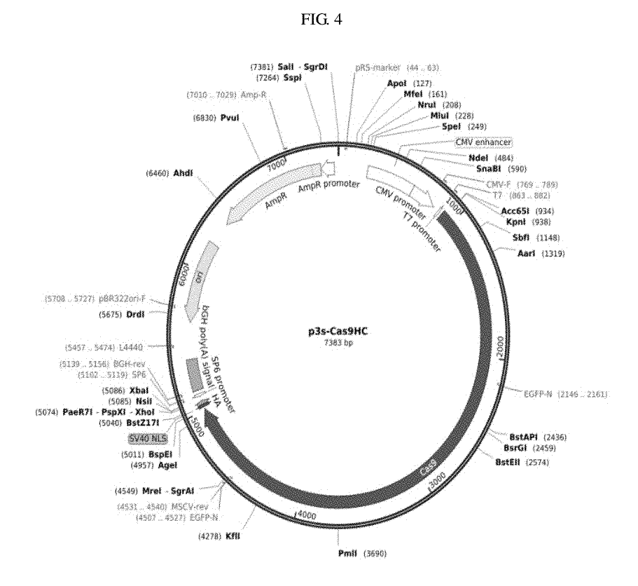

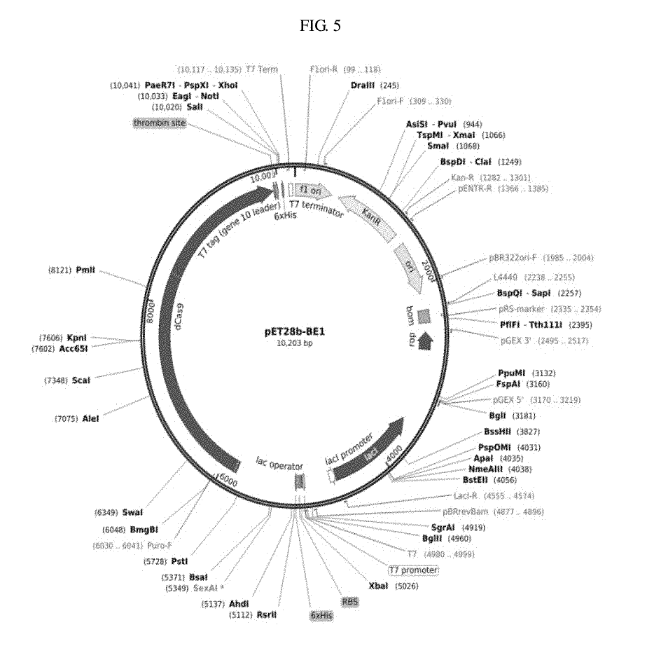

HEK293T cells (ATCC CRL-11268) were maintained in DMEM (Dulbecco Modified Eagle Medium) supplemented with 10% (w/v) FBS and 1% (w/v) penicillin/streptomycin (Welgene). HEK293T cells (1.5×10⁵) were seeded on 24-well plates and transfected at ˜80% confluency with sgRNA plasmid (500 ng) and Base Editor plasmid (Addgene plasmid #73019 (Expresses BE1 with C-terminal NLS in mammalian cells; rAPOBEC1-XTEN-dCas9-NLS; FIG. 3a), #73020 (Expresses BE2 in mammalian cells; rAPOBEC1-XTEN-dCas9-UGI-NLS; FIG. 3b), #73021 (Expresses BE3 in mammalian cells; rAPOBEC1-XTEN-Cas9n-UGI-NLS; FIG. 3c)) (1.5 μg) or Cas9 expression plasmid (Addgene plasmid #43945; FIG. 4), using Lipofectamine 2000 (Invitrogen). Genomic DNA was isolated using DNeasy Blood & Tissue Kit (Qiagen) at 72 hours after transfection. The cells were not tested for mycoplasma contamination.

The sgRNA used in the following Examples was constructed by converting T to U on the overall sequence at an on-target site (on-target sequence; EMX1 on-target sequence; GAGTCCGAGCAGAAGAAGAAGGG (SEQ ID NO: 14)), except the 5′-terminal PAM sequence ((5′-NGG-3′) wherein N is A, T, G, or C), and employing the converted sequence as the targeting sequence ‘(N_{ca9})₁’ of the following General Formula 3:

5(_{Ncas9})ₗ-(GUUUUAGAGCUA; SEQ ID NO: 1)-(GAAA)-
(UAGCAAGUUAAAAUAAGGCUAGUCCGUUAUCAACUUGAAAAAGUGGCAC
CGAGUCGGUGC; SEQ ID NO: 3)-3 (General Formula 3;
oligonucleotide linker: GAAA).

2. Protein Purification

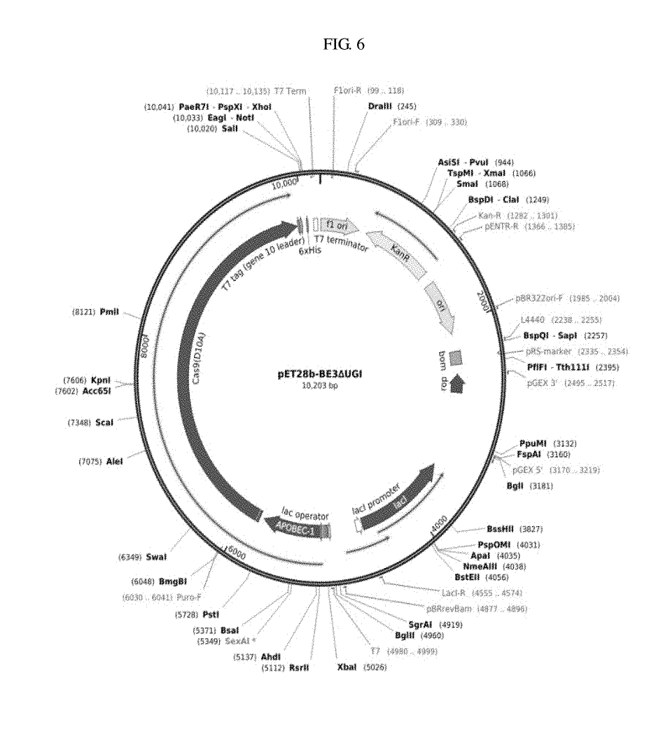

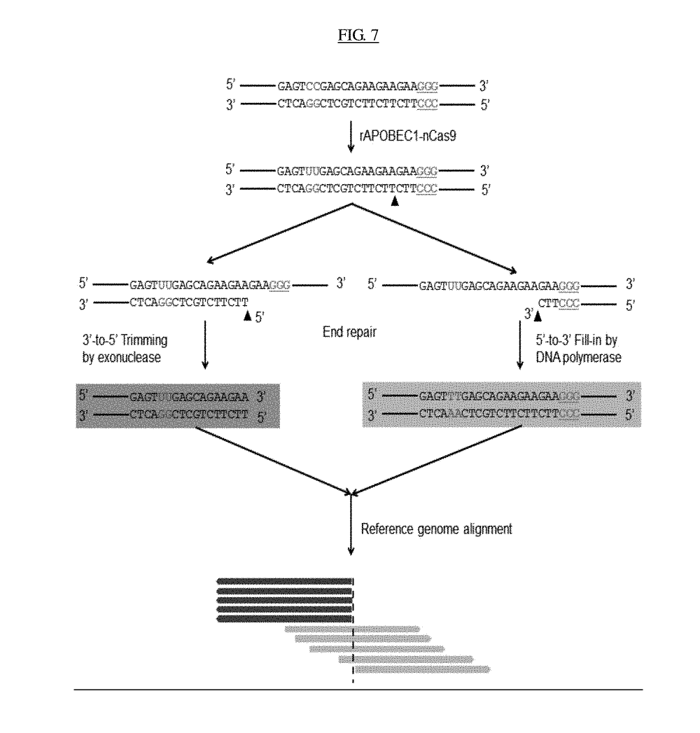

The His6-rAPOBEC1-XTEN-dCas9 protein-encoding plasmid (pET28b-BE1; Expresses BE1 with N-terminal His6 tag in E. coli; FIG. 5) was generously given by David Liu (Addgene plasmid #73018). The His6-rAPOBEC1-XTEN-dCas9 protein-encoding plasmid pET28b-BE1 was converted into a His6-rAPOBEC1-nCas9 protein (BE3 delta UGI; BE3 variant lacking a UGI domain) encoding plasmid (pET28b-BE3 delta UGI; FIG. 6) by site directed mutagenesis for substituting A840 with H840 in the dCas9.

Rosetta expression cells (Novagen, catalog number: 70954-3CN) were transformed with the prepared pET28b-BE1 or pET28b-BE3 delta UGI and cultured overnight in Luria-Bertani (LB) broth containing 100 μg/ml kanamycin and 50 mg/ml carbenicilin at 37° C. Ten ml of the overnight cultures of Rosetta cells containing pET28b-BE1 or pET28b-BE3 delta UGI was inoculated into 400 ml LB broth containing 100 μg/ml kanamycin and 50 mg/ml carbenicilin and cultured at 30° C. until the OD600 reached 0.5-0.6. The cells were cooled to 16° C. for 1 hour, supplemented with 0.5 mM IPTG (Isopropyl β-D-1-thiogalactopyranoside), and cultured for 14-18 hours.

For protein purification, cells were harvested by centrifugation at 5,000×g for 10 min at 4° C. and lysed by sonication in 5 ml lysis buffer (50 mM NaH₂PO₄, 300 mM NaCl, 1 mM DTT, and 10 mM imidazole, pH 8.0) supplemented with lysozyme (Sigma) and a protease inhibitor (Roche complete, EDTA-free). The soluble lysate obtained after centrifugation of the cell lysis mixture at 13,000 rpm. for 30 min at 4° C. was incubated with Ni-NTA agarose resin (Qiagen) for 1 hour at 4° C. The cell lysate/Ni-NTA mixture was applied to a column and washed with a buffer (50 mM NaH₂PO₄, 300 mM NaCl, and 20 mM imidazole, pH 8.0). The BE3 protein was eluted with an elution buffer (50 mM NaH₂PO₄, 300 mM NaCl, and 250 mM imidazole, pH 8.0). The eluted protein was buffer exchanged with a storage buffer (20 mM HEPES-KOH (pH 7.5), 150 mM KCl, 1 mM DTT, and 20% glycerol) and concentrated with centrifugal filter units (Millipore) to give purified rAPOBEC1-nCas9 protein.

3. Deamination of Genomic DNA

Genomic DNA was purified (extracted) from HEK293T cells with a DNeasy Blood & Tissue Kit (Qiagen) according to the manufacturer's instructions. Genomic DNA (10 μg) was incubated with the rAPOBEC1-nCas9 protein (300 nM) purified in Reference Example 2 and an sgRNA (900 nM) in a reaction volume of 500 μL for 8 hours at 37° C. in a buffer (100 mM NaCl, 40 mM Tris-HCl, 10 mM MgCl₂, and 100 μg/ml BSA, pH 7.9).

The used sgRNA was constructed by converting T to U on the overall sequence at an on-target site (on-target sequence; EMX1 on-target sequence; GAGTCCGAGCAGAAGAAGAAGGG (SEQ ID NO: 14)), except the 5′-terminal PAM sequence ((5′-NGG-3′) wherein N is A, T, G, or C), and employing the converted sequence as the targeting sequence ‘(N_{cas9})₁’ of the following General Formula 3:

5-(N_{cas9})ₗ-(GUUUUAGAGCUA)-(GAAA)-
(UAGCAAGUUAAAAUAAGGCUAGUCCGUUAUCAACUUGAAAAAGUGGCAC
CGAGUCGGUGC)-3′ (General Formula 3; oligonucleotide
linker: GAAA).

After removal of sgRNA using RNase A (50 μg/mL), uracil-containing genomic DNA was purified with a DNeasy Blood & Tissue Kit (Qiagen). The on-target site was amplified by PCR using a SUN-PCR blend and subjected to Sanger sequencing to check BE3-mediated cytosine deamination and USER-mediated DNA cleavage.

4. Whole Genome and Digenome Sequencing

Genomic DNA (1 μg) was fragmented to the 400- to 500-bp range using the Covaris system (Life Technologies) and blunt-ended using End Repair Mix (Thermo Fischer). Fragmented DNA was ligated with adapters to produce libraries, which were then subjected to WGS (whole genome sequencing) using HiSeq X Ten Sequencer (Illumina) at Macrogen. (Kim, D., Kim, S., Kim, S., Park, J. & Kim, J. S. Genome-wide target specificities of CRISPR-Cas9 nucleases revealed by multiplex Digenome-seq. Genome research 26, 406-415 (2016)).

5. Targeted Deep Sequencing

On-target and potential off-target sites were amplified with a KAPA HiFi HotStart PCR kit (KAPA Biosystems #KK2501) for deep sequencing library generation. Pooled PCR amplicons were sequenced using MiniSeq (Illumina) or Illumina Miseq (LAS Inc. Korea) with TruSeq HT Dual Index system (Illumina).

Primers used in the targeted deep sequencing are as follows:

EMX1
On-target sequence:
(SEQ ID NO: 14)
GAGTCCGAGCAGAAGAAGAAGGG
1^{st} PCR
Forward (5′→3′):
(SEQ ID NO: 15)
AGTGTTGAGGCCCCAGTG;
Reverse (5′→3′):
(SEQ ID NO: 16)
GTGACTGGAGTTCAGACGTGTGCTCTTCCGATCTCAGCAGCAAGCAG
CACTCT;
2^{nd} PCR
Forward (5′→3′):
(SEQ ID NO: 17)
ACACTCTTTCCCTACACGACGCTCTTCCGATCTGGGCCTCCTGAGTTT
CTCAT;
Reverse (5′→3′)
(SEQ ID NO: 18)
GTGACTGGAGTTCAGACGTGTGCTCTTCCGATCTCAGCAGCAAGCAG
CACTCT.

### Example 1

### Identification of BE3 Off-Target Site using Digenome-seq

A human genomic DNA was in vitro treated with the ribonucleic acid protein in which EMX1-specific sgRNA (see Reference Example 3; on-target sequence: SEQ ID NO: 14) is complexed with rAPOBEC1-nCas9 protein (BE3: purified in Reference Example 2), to induce C→U conversion on one strand and nick formation on the other strand at on-target and off-target sites, followed by performing Digenome-seq with reference to Reference Example 4. In this Example, neither Uracil DNA glycosylase (UDG) nor DNA glycosylase-lyase Endonuclease VIII were used. After end repair and adaptor ligation, the BE3-treated genomic DNA was subjected to whole genome sequencing (WGS).

The procedure is schematically depicted in FIG. 7.

Uniform alignment of sequence reads in one strand and on-target sites of C→U conversion in the other strand, and off-target sites were computationally identified.

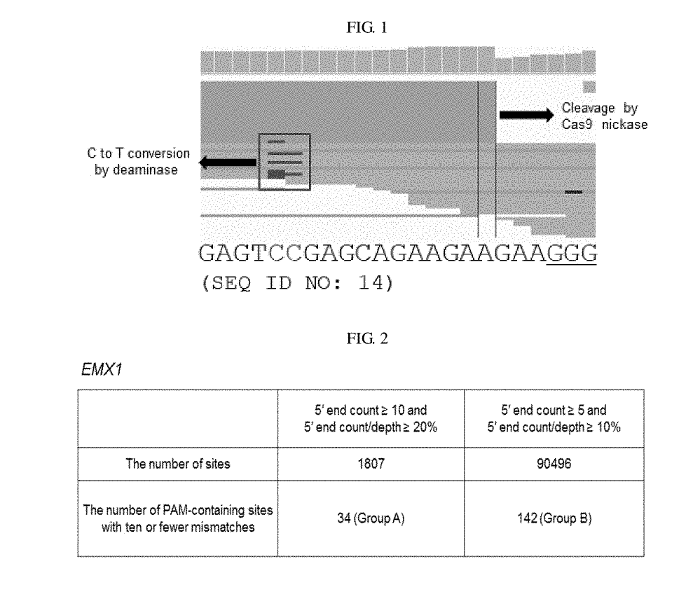

FIG. 1 is a representative IGV image showing straight alignments of sequence reads at the EMX1 on-target site.

In FIG. 2, the number of nicked sites at which sequence reads have uniform alignment only one strand obtained as a result of the Digenome-seq (sites (reads) at which the 5′ ends have straight alignment) and the number of PAM-containing sites with 10 or fewer mismatches among the sites are given. Groups A and B were identified to have absolute numbers (n≥10 and 5, respectively) and relative numbers (20% and 10%, respectively)

The absolute number (n≥5 or 10) and relative number (10% or 20%) of sequence reads with the same 5′ ends were counted over the entire human genome to enumerate all sites relevant to uniform alignment patterns in genome. In result, as shown in FIGS. 2, 90,496 and 1,807 corresponding sites were acquired. Of the single-strand nicked sites, 34 (Group A) and 142 (Group B; inclusive of Group A) each have a 3-bp PAM (5′-NGN-3′ or 5′-NNG-3′) downstream of the single-strand nicked sites and show homology to the EMX1 target sequence to an extent of 10 or fewer mismatches.

The Cas9-induced indel frequency and BE3-induced substitution frequency at BE3 off-target sites for EMX1, identified by Digenome-seq, were measured using targeted deep sequencing (see Reference Example 5) in HEK293T cells. From WGS data obtained using intact genomic DNA and rAPOBEC1-nCas9-treated genomic DNA, C→T conversion at each of their sites were investigated.

DNA sequences cleaved by BE3 in EMX1 (1 on-target site+141 off-target sites=142 sites in total) are summarized in Table 1, below (in Table 1, on-target sequences and mismatched bases are expressed in lower cases).

TABLE 1

| | | | | SEQ ID |
|---|---|---|---|---|
| | Chr | Position | DNA seq at a nickase sites | NO: |
| EMX1-001 | chr2 | 73160998 | GAGTCCGAGCAGAAGAAGAAGGG | 14 |
| (on- target) | | | | |
| EMX1-002 | chr4 | 131662222 | GAaTCCaAG-AGAAGAAGAATGG | 19 |
| EMX1-003 | chr2 | 219845072 | GAGgCCGAGCAGAAGAAagACGG | 20 |
| EMX1-004 | chr11 | 62365273 | GAaTCCaAGCAGAAGAAGAgAAG | 21 |
| EMX1-005 | chr8 | 128801258 | GAGTCCtAGCAGgAGAAGAAGAG | 22 |
| EMX1-006 | chr15 | 44109763 | GAGTCtaAGCAGAAGAAGAAGAG | 23 |
| EMX1-007 | chr19 | 24250503 | GAGTCCaAGCAGtAGAgGAAGGG | 24 |
| EMX1-008 | chr6 | 9118799 | acGTCtGAGCAGAAGAAGAATGG | 25 |
| EMX1-009 | chr5 | 9227162 | aAGTCtGAGCAcAAGAAGAATGG | 26 |
| EMX1-010 | chr1 | 4515013 | GtGTCCtAG-AGAAGAAGAAGGG | 27 |
| EMX1-011 | chr5 | 45359067 | GAGTtaGAGCAGAAGAAGAAAGG | 28 |
| EMX1-012 | chr13 | 96928092 | GAGaCaGAG-AGAAGAAGAATGG | 29 |
| EMX1-013 | chr18 | 34906762 | GAGcCtGAGCgGAAGAgGAAAGG | 30 |
| EMX1-014 | chr1 | 184236243 | aAtaCaGAGCAGAAGAAGAATGG | 31 |
| EMX1-015 | chr18 | 1677040 | agtcCaGAGCAaAAtAAGAAGGG | 32 |
| EMX1-016 | chr1 | 33606480 | GAGcCtGAGCAGAAGgAGAAGGG | 33 |
| EMX1-017 | chr3 | 111296327 | GAagaaGAGCAaAAGAAGAAGGG | 34 |
| EMX1-018 | chr22 | 34716275 | GtGaCaGAGCAaAAGAAGAAAGG | 35 |
| EMX1-019 | chr3 | 37781974 | GAagagGAGCAaAAGAAGAAGGG | 36 |
| EMX1-020 | chr20 | 6653999 | aAGTCCagaCAGAAGAAGAAGGA | 37 |
| EMX1-021 | chr16 | 78848850 | aAaTCCaAcCAGAAGAAGAAAGG | 38 |
| EMX1-022 | chr6 | 92449690 | GttcaaGAGCAGgAGAAGAAGGG | 39 |
| EMX1-023 | chr4 | 87256692 | GAGTaaGAGaAGAAGAAGAAGGG | 40 |
| EMX1-024 | chr11 | 43747948 | aAGcCCGAGCAaAgGAAGAAAGG | 41 |
| EMX1-025 | chr5 | 160643032 | cctataGAGCAaAAGAAGAAAGG | 42 |
| EMX1-026 | chr11 | 120873098 | GAtcaaGAGaAGAAGAAGAAGGG | 43 |
| EMX1-027 | chr5 | 62692054 | cAaaaaGAGCAaAAGAAGAACGG | 44 |
| EMX1-028 | chrX | 3077291 | tAcagtGAGCAaAAGAAGAAGGG | 45 |
| EMX1-029 | chr14 | 98236084 | GttcaaGAGCAGgAGAAGAAGGG | 46 |
| EMX1-030 | chr2 | 205473563 | ttcTCaGAGCAaAAGAAGAATGG | 47 |
| EMX1-031 | chr3 | 189633259 | cttTGCcAGGAGAAGgAcAtTGC | 48 |
| EMX1-032 | chr10 | 58498683 | agGTtaGAGCAaAAGAAGAAAGG | 49 |
| EMX1-033 | chr1 | 35818892 | tAtaCgGAGCAGAAGAAGAATGG | 50 |
| EMX1-034 | chr3 | 45605387 | GAGTCCacaCAGAAGAAGAAAGA | 51 |
| EMX1-035 | chr3 | 5031614 | GAaTCCaAGCAGgAGAAGAAGGA | 52 |
| EMX1-036 | chr12 | 106646090 | aAGTCCatGCAGAAGAgGAAGGG | 53 |
| EMX1-037 | chr1 | 23720618 | aAGTCCGAGgAGAgGAAGAAAGG | 54 |
| EMX1-038 | chr11 | 107812992 | aAGTCCaAGt-GAAGAAGAAAGG | 55 |
| EMX1-039 | chr4 | 169444372 | GAGaaCGAGaAGAAagAGgAGAG | 56 |
| EMX1-040 | chr6 | 18327737 | GAGagaGAGagagAGAgGgAGGG | 57 |
| EMX1-041 | chr2 | 230161576 | ctGgCaGAGCAaAAGAAGAgGGG | 58 |
| EMX1-042 | chr3 | 95690186 | tcaTCCaAGCAGAAGAAGAAGAG | 59 |
| EMX1-043 | chr4 | 33321466 | GtacagGAGCAGgAGAAGAATGG | 60 |
| EMX1-044 | chr22 | 49900715 | aAGaagGAGaAGAAGAAGAAGGG | 61 |
| EMX1-045 | chr12 | 94591214 | GAGagaGAGagagAGAgaAAGGG | 62 |
| EMX1-046 | chr5 | 146833190 | GAGcCgGAGCAGAAGAAGgAGGG | 63 |
| EMX1-047 | chr6 | 111509461 | GAGggaGAGagGgAGAgagAAAG | 64 |
| EMX1-048 | chr1 | 26490139 | ttaTCtccGagaAgGAAGAAGGG | 65 |
| EMX1-049 | chr6 | 31265461 | GAtTCtGtcCcGAAtcAGAAGGG | 66 |
| EMX1-050 | chr14 | 30099303 | atGcaaGAGaAGAAGAAGAAAGG | 67 |
| EMX1-051 | chr3 | 83057859 | agcaggGAGCAGAgGAAGAATGG | 68 |
| EMX1-052 | chr15 | 35575311 | GAGaagGAGaAGAAGAAGAAGGG | 69 |
| EMX1-053 | chr1 | 55846672 | actctaGAGCAGAAaAAGAATGG | 70 |
| EMX1-054 | chr6 | 104384459 | GAGgagGAGgAGgAGgAaggAGG | 71 |
| EMX1-055 | chr19 | 9975831 | aAagagGAGaAGAAGAAGAAGGG | 72 |
| EMX1-056 | chr12 | 99525769 | GgGgagGAGCAGAAGAAGAgAGG | 73 |
| EMX1-057 | chr6 | 162280006 | agGcCgagGCAGgAGAAtAgGAG | 74 |
| EMX1-058 | chr7 | 85359110 | GAGaagGAGCAGAAaAAGAATGG | 75 |
| EMX1-059 | chr2 | 10462867 | acagtaGAGCAGAAGAAGAcTGG | 76 |
| EMX1-060 | chr3 | 18195303 | atccaaGAGCAGgAGAAGAAGGG | 77 |
| EMX1-061 | chr2 | 57855994 | ataagaGAGCAaAAGAAGAAAGG | 78 |
| EMX1-062 | chr6 | 33957284 | GAGagaGAGagagAGAgaAACGG | 79 |
| EMX1-063 | chr22 | 37474903 | GAGaagGAGaAGAAGgAGAAGAG | 80 |
| EMX1-064 | chr8 | 141193983 | aAGaagaAGaAGAAGAAGAAGAG | 81 |
| EMX1-065 | chr1 | 110038435 | thcggGAGCAGAAGAAGAACAG | 82 |
| EMX1-066 | chr4 | 117483357 | atcaCaGAGCAGgAGAAGAAGGG | 83 |
| EMX1-067 | chr4 | 6150362 | aAacagGAGCAGAgGAAGAAGGG | 84 |
| EMX1-068 | chr2 | 116142148 | aAGaagagGaAGAgGAgGAAAAG | 85 |
| EMX1-069 | chr12 | 30794309 | GAaatgGAGaAGAAGAAGAAGGG | 86 |
| EMX1-070 | chr22 | 44527016 | GAGagaGAaagaAAGAAaAAGGA | 87 |
| EMX1-071 | chr9 | 96189722 | GctgtgGAGCAaAAGAAGAAAGG | 88 |
| EMX1-072 | chr8 | 113493465 | GAGgagGAGCAGAAGAAGAAAAG | 89 |
| EMX1-073 | chr11 | 46171476 | tAaaagGAGCAGAAaAAGAAGGG | 90 |
| EMX1-074 | chrX | 3075272 | tAccttGAGCAaAAGAAGAAGGG | 91 |
| EMX1-075 | chr5 | 56038567 | aAGaagGAGaAGAAGAAGAAGGG | 92 |
| EMX1-076 | chr2 | 71789100 | GcaggaGAGCAGAAGAAGAAAGG | 93 |
| EMX1-077 | chr7 | 52389195 | aAGagCGAGattAAGAgGAATGG | 94 |
| EMX1-078 | chr5 | 31088930 | aAGaaaGgagAGgAGAgGAgAGG | 95 |
| EMX1-079 | chr11 | 111680806 | agtagtGAGCAGAAGAAGAtAGG | 96 |
| EMX1-080 | chr20 | 51306677 | aAGaagGAGaAGAAGAAGAAGAG | 97 |
| EMX1-081 | chr19 | 38433655 | GAGagaGAGagagAGAgaAAGAG | 98 |
| EMX1-082 | chr8 | 60956107 | GgccagGAGCAGgAGAAGAAGGG | 99 |
| EMX1-083 | chr16 | 26617803 | agaggaGAGCAGAAGAAGgATGG | 100 |
| EMX1-084 | chr12 | 52621931 | aAGaagGAGaAGAAGAAGgAGGA | 101 |
| EMX1-085 | chr3 | 156028864 | cAtTaaGAGCAGgAGAAGAAGGG | 102 |
| EMX1-086 | chr6 | 40280504 | cgcTgatAcagaAAGAAGAATGG | 103 |
| EMX1-087 | chr1 | 35385601 | GAagtgGAGCAGgAGAAGAAGGG | 104 |
| EMX1-088 | chr1 | 59299359 | tttgtgGAGCAGAAaAAGAAAGG | 105 |
| EMX1-089 | chr15 | 61646877 | aAGTCaGAGgAGAAGAAGAAGGG | 106 |
| EMX1-090 | chr2 | 159685754 | aAagCtGAGCAGAAaAAGAAGGG | 107 |
| EMX1-091 | chr12 | 41494108 | GcagtgGAGCAGAAGAAGAtGGG | 108 |
| EMX1-092 | chr7 | 119831026 | acaaaaGAGCAGAgGAAGAAAGG | 109 |
| EMX1-093 | chr1 | 234492864 | GAagtaGAGCAGAAGAAGAAGCG | 110 |
| EMX1-094 | chr14 | 104091588 | aAagagGgagAGAAGAAGAAGGG | 111 |
| EMX1-095 | chr1 | 31954326 | aAGaagGAGaAGAAGAAGAAGAG | 112 |
| EMX1-096 | chr8 | 120587501 | aAGgCCaAGCAGAAGAgtAATGG | 113 |
| EMX1-097 | chr2 | 46020469 | acacaaGAGCAGAAGAAGAAAGA | 114 |
| EMX1-098 | chr2 | 219294645 | GccaatGAGCAGgAGAAGAAGGG | 115 |
| EMX1-099 | chr8 | 11924153 | cAtataGAGCAaAAGAAGAgAGG | 116 |
| EMX1-100 | chr6 | 54740531 | GAGgtgGAGggGAAGAgGgAAGG | 117 |
| EMX1-101 | chr1 | 156786840 | GAGagaGAGagagAGAgaAAGGG | 118 |
| EMX1-102 | chr6 | 30791217 | aAGgagGAGaAGAAGAAGAAGGG | 119 |
| EMX1-103 | chr3 | 192777993 | GAGggaGAGagagAGAgagAAAG | 120 |
| EMX1-104 | chr2 | 36207879 | agtcggGAGCAGgAGAAGAAAGG | 121 |
| EMX1-105 | chr16 | 54831367 | GttcaaGAGCAGAAGAAGAATGG | 122 |
| EMX1-106 | chr6 | 160868147 | tctaaaGAGCAGAAaAAGAAAGG | 123 |
| EMX1-107 | chr2 | 24438043 | actgatGAGCAGAAGAAGAAAGG | 124 |
| EMX1-108 | chr22 | 37102243 | aAGaagGAGaAGAAGAAGgAGGA | 125 |
| EMX1-109 | chr11 | 121786535 | agGaaaagagAGAAGAAGAAGGG | 126 |
| EMX1-110 | chr7 | 3337380 | GAGgagGAGaAGAAGAAGAAGGG | 127 |
| EMX1-111 | chr8 | 112924257 | GAGagaGAGagagAGAgaAAGGG | 128 |
| EMX1-112 | chr16 | 69047289 | GAGgCCGAagctgAGgtGggAGG | 129 |
| EMX1-113 | chr8 | 105164125 | GAGcCCaAGaAGAAGAAGAAGGA | 130 |
| EMX1-114 | chr13 | 83353702 | atGTaCagagAGAAGAAGAAAGG | 131 |
| EMX1-115 | chr2 | 102929260 | GccTtCagagAGAAGAAGAATGG | 132 |
| EMX1-116 | chr15 | 22366621 | GgagtaGAGCAGAgGAAGAAGGG | 133 |
| EMX1-117 | chr2 | 172374203 | GAagtaGAGCAGAAGAAGAAGCG | 134 |
| EMX1-118 | chr8 | 31096390 | GctcCtGAGCAGAAGAAGAACAG | 135 |
| EMX1-119 | chr2 | 66729772 | agtTCaGAGCAGgAGAAGAATGG | 136 |
| EMX1-120 | chr2 | 14472327 | atGaaCagagAGAAGAAGAATGG | 137 |
| EMX1-121 | chr8 | 140468447 | GAGagCGAGagagAGAgagAGGG | 138 |
| EMX1-122 | chr7 | 52204863 | aAaaagGAGCAGAAGAAGAAGGA | 139 |
| EMX1-123 | chr1 | 151027598 | ttcTCCaAGCAGAAGAAGAAGAG | 140 |
| EMX1-124 | chr1 | 35590719 | GAGagaGAGagagAGAgaAAGGG | 141 |
| EMX1-125 | chr1 | 106744880 | ttGgaaagagAGAAGAAGAAGGG | 142 |
| EMX1-126 | chr10 | 115484209 | aAGaggaAGaAGAAGAAGAAGAG | 143 |
| EMX1-127 | chr3 | 119686684 | GAGagaGAGaAagAGAAagAGAG | 144 |
| EMX1-128 | chr8 | 53295601 | GAagaaGAGaAGAAGAAGAAGGG | 145 |
| EMX1-129 | chr18 | 12032247 | GAtTCtGAGaAaAttAAGAtGGG | 146 |
| EMX1-130 | chr15 | 61383748 | GgGctCcgGCAGAAGAtGccATG | 147 |
| EMX1-131 | chr1 | 209298672 | GAtTCCaAGCAatgGAgGAgGGG | 148 |
| EMX1-132 | chr7 | 17446438 | GtccaaGAGCAGgAGAAGAAGGG | 149 |
| EMX1-133 | chr13 | 74473871 | atcTggGAGCAGgAGAAGAAGGG | 150 |
| EMX1-134 | chr5 | 5141237 | GAGgatccGagGAtGtAGAAGGG | 151 |
| EMX1-135 | chr12 | 5041728 | GAagaaGAagAaAgaAAGAAAGA | 152 |
| EMX1-136 | chr8 | 112756160 | cAGagaGAGaAtAAGtAGcATAG | 153 |
| EMX1-137 | chr8 | 17384135 | tgaggaagagAGAAGAAGAAAGG | 154 |
| EMX1-138 | chr12 | 4545932 | cAagCatgagAGAAGAAGAtGGG | 155 |
| EMX1-139 | chr10 | 58848728 | GAGcaCGAGCAagAGAAGAAGGG | 156 |
| EMX1-140 | chr14 | 48932119 | GAGTCCcAGCAaAAGAAGAAAAG | 157 |
| EMX1-141 | chr3 | 145057362 | GAGTCCct-CAGgAGAAGAAAGG | 158 |
| EMX1-142 | chr9 | 111348573 | GAGTCCttG-AGAAGAAGgAAGG | 159 |

Counts (numbers of sequence reads having the same 5′ end), depths (numbers of sequence reads at specific sites), % (count/depth), and counts of reads with C→T conversion, which were all measured at the nicked sites enumerated in Table 1, are summarized in Table 2, below:

TABLE 2

| | | | % (count/ | C to T conversion | | | |
|---|---|---|---|---|---|---|---|
| | count | depth | depth) | (+) Base editor | Untreated | Group A | Group B |
| EMX1-001 | 21 | 51 | 41.2 | 6 | 0 | v | v |
| (on-target) | | | | | | | |
| EMX1-002 | 21 | 39 | 53.8 | 8 | 0 | v | v |
| EMX1-003 | 22 | 41 | 53.7 | 0 | 0 | v | v |
| EMX1-004 | 36 | 79 | 45.6 | 10 | 0 | v | v |
| EMX1-005 | 29 | 68 | 42.6 | 1 | 0 | v | v |
| EMX1-006 | 26 | 62 | 41.9 | 9 | 0 | v | v |
| EMX1-007 | 10 | 29 | 34.5 | 0 | 0 | v | v |
| EMX1-008 | 24 | 86 | 27.9 | 0 | 0 | v | v |
| EMX1-009 | 44 | 159 | 27.7 | 10 | 0 | v | v |
| EMX1-010 | 11 | 41 | 26.8 | 0 | 0 | v | v |
| EMX1-011 | 50 | 109 | 45.9 | N.A. | N.A. | v | v |
| EMX1-012 | 15 | 43 | 34.9 | 1 | 0 | v | v |
| EMX1-013 | 16 | 46 | 34.8 | 0 | 0 | v | v |
| EMX1-014 | 22 | 64 | 34.4 | 0 | 1 | v | v |
| EMX1-015 | 16 | 53 | 30.2 | 0 | 0 | v | v |
| EMX1-016 | 19 | 63 | 30.2 | 1 | 0 | v | v |
| EMX1-017 | 24 | 82 | 29.3 | N.A. | N.A. | v | v |
| EMX1-018 | 24 | 85 | 28.2 | 0 | 0 | v | v |
| EMX1-019 | 14 | 50 | 28.0 | N.A. | N.A. | v | v |
| EMX1-020 | 10 | 36 | 27.8 | 0 | 0 | v | v |
| EMX1-021 | 13 | 47 | 27.7 | 0 | 0 | v | v |
| EMX1-022 | 13 | 48 | 27.1 | 1 | 0 | v | v |
| EMX1-023 | 10 | 37 | 27.0 | N.A. | N.A. | v | v |
| EMX1-024 | 11 | 42 | 26.2 | 0 | 0 | v | v |
| EMX1-025 | 15 | 58 | 25.9 | N.A. | N.A. | v | v |
| EMX1-026 | 11 | 43 | 25.6 | 0 | 0 | v | v |
| EMX1-027 | 16 | 67 | 23.9 | N.A. | N.A. | v | v |
| EMX1-028 | 10 | 44 | 22.7 | N.A. | N.A. | v | v |
| EMX1-029 | 10 | 45 | 22.2 | 0 | 0 | v | v |
| EMX1-030 | 14 | 63 | 22.2 | 0 | 0 | v | v |
| EMX1-031 | 13 | 61 | 21.3 | 0 | 0 | v | v |
| EMX1-032 | 13 | 61 | 21.3 | N.A. | N.A. | v | v |
| EMX1-033 | 14 | 66 | 21.2 | 0 | 0 | v | v |
| EMX1-034 | 14 | 53 | 26.4 | 2 | 0 | v | v |
| EMX1-035 | 9 | 48 | 18.8 | 0 | 0 | — | v |
| EMX1-036 | 8 | 46 | 17.4 | 1 | 0 | — | v |
| EMX1-037 | 8 | 51 | 15.7 | 0 | 0 | — | v |
| EMX1-038 | 6 | 42 | 14.3 | 0 | 0 | — | v |
| EMX1-039 | 7 | 22 | 31.8 | 1 | 0 | — | v |
| EMX1-040 | 7 | 22 | 31.8 | N.A. | N.A. | — | v |
| EMX1-041 | 7 | 23 | 30.4 | 0 | 0 | — | v |
| EMX1-042 | 7 | 25 | 28.0 | 0 | 0 | — | v |
| EMX1-043 | 6 | 23 | 26.1 | 0 | 0 | — | v |
| EMX1-044 | 7 | 27 | 25.9 | N.A. | N.A. | — | v |
| EMX1-045 | 8 | 35 | 22.9 | N.A. | N.A. | — | v |
| EMX1-046 | 9 | 40 | 22.5 | 0 | 0 | — | v |
| EMX1-047 | 8 | 38 | 21.1 | N.A. | N.A. | — | v |
| EMX1-048 | 5 | 24 | 20.8 | 0 | 0 | — | v |
| EMX1-049 | 7 | 34 | 20.6 | 0 | 0 | — | v |
| EMX1-050 | 8 | 40 | 20.0 | 0 | 0 | — | v |
| EMX1-051 | 6 | 30 | 20.0 | N.A. | N.A. | — | v |
| EMX1-052 | 10 | 51 | 19.6 | N.A. | N.A. | — | v |
| EMX1-053 | 12 | 63 | 19.0 | 0 | 0 | — | v |
| EMX1-054 | 7 | 37 | 18.9 | N.A. | N.A. | — | v |
| EMX1-055 | 12 | 64 | 18.8 | N.A. | N.A. | — | v |
| EMX1-056 | 8 | 43 | 18.6 | N.A. | N.A. | — | v |
| EMX1-057 | 5 | 27 | 18.5 | 1 | 0 | — | v |
| EMX1-058 | 9 | 49 | 18.4 | N.A. | N.A. | — | v |
| EMX1-059 | 13 | 71 | 18.3 | N.A. | N.A. | — | v |
| EMX1-060 | 10 | 55 | 18.2 | 0 | 0 | — | v |
| EMX1-061 | 10 | 55 | 18.2 | N.A. | N.A. | — | v |
| EMX1-062 | 5 | 28 | 17.9 | N.A. | N.A. | — | v |
| EMX1-063 | 5 | 28 | 17.9 | N.A. | N.A. | — | v |
| EMX1-064 | 7 | 40 | 17.5 | N.A. | N.A. | — | v |
| EMX1-065 | 13 | 76 | 17.1 | 0 | 0 | — | v |
| EMX1-066 | 5 | 30 | 16.7 | 0 | 0 | — | v |
| EMX1-067 | 5 | 30 | 16.7 | 0 | 0 | — | v |
| EMX1-068 | 6 | 36 | 16.7 | N.A. | N.A. | — | v |
| EMX1-069 | 19 | 115 | 16.5 | N.A. | N.A. | — | v |
| EMX1-070 | 6 | 37 | 16.2 | N.A. | N.A. | — | v |
| EMX1-071 | 9 | 56 | 16.1 | N.A. | N.A. | — | v |
| EMX1-072 | 15 | 94 | 16.0 | N.A. | N.A. | — | v |
| EMX1-073 | 11 | 70 | 15.7 | N.A. | N.A. | — | v |
| EMX1-074 | 7 | 45 | 15.6 | 0 | 0 | — | v |
| EMX1-075 | 9 | 59 | 15.3 | N.A. | N.A. | — | v |
| EMX1-076 | 9 | 59 | 15.3 | N.A. | N.A. | — | v |
| EMX1-077 | 5 | 33 | 15.2 | 0 | 0 | — | v |
| EMX1-078 | 14 | 93 | 15.1 | N.A. | N.A. | — | v |
| EMX1-079 | 6 | 40 | 15.0 | N.A. | N.A. | — | v |
| EMX1-080 | 11 | 75 | 14.7 | N.A. | N.A. | — | v |
| EMX1-081 | 6 | 42 | 14.3 | N.A. | N.A. | — | v |
| EMX1-082 | 6 | 43 | 14.0 | 0 | 0 | — | v |
| EMX1-083 | 6 | 43 | 14.0 | N.A. | N.A. | — | v |
| EMX1-084 | 7 | 50 | 14.0 | N.A. | N.A. | — | v |
| EMX1-085 | 7 | 50 | 14.0 | N.A. | N.A. | — | v |
| EMX1-086 | 5 | 36 | 13.9 | N.A. | N.A. | — | v |
| EMX1-087 | 7 | 51 | 13.7 | N.A. | N.A. | — | v |
| EMX1-088 | 7 | 51 | 13.7 | N.A. | N.A. | — | v |
| EMX1-089 | 6 | 44 | 13.6 | 0 | 0 | — | v |
| EMX1-090 | 10 | 74 | 13.5 | 0 | 0 | — | v |
| EMX1-091 | 12 | 89 | 13.5 | N.A. | N.A. | — | v |
| EMX1-092 | 5 | 37 | 13.5 | N.A. | N.A. | — | v |
| EMX1-093 | 7 | 52 | 13.5 | N.A. | N.A. | — | v |
| EMX1-094 | 6 | 45 | 13.3 | N.A. | N.A. | — | v |
| EMX1-095 | 6 | 46 | 13.0 | N.A. | N.A. | — | v |
| EMX1-096 | 11 | 85 | 12.9 | 0 | 0 | — | v |
| EMX1-097 | 6 | 47 | 12.8 | 0 | 0 | — | v |
| EMX1-098 | 5 | 39 | 12.8 | N.A. | N.A. | — | v |
| EMX1-099 | 6 | 48 | 12.5 | N.A. | N.A. | — | v |
| EMX1-100 | 6 | 48 | 12.5 | N.A. | N.A. | — | v |
| EMX1-101 | 8 | 64 | 12.5 | N.A. | N.A. | — | v |
| EMX1-102 | 7 | 57 | 12.3 | N.A. | N.A. | — | v |
| EMX1-103 | 6 | 50 | 12.0 | N.A. | N.A. | — | v |
| EMX1-104 | 7 | 59 | 11.9 | 0 | 0 | — | v |
| EMX1-105 | 6 | 51 | 11.8 | 0 | 0 | — | v |
| EMX1-106 | 9 | 77 | 11.7 | N.A. | N.A. | — | v |
| EMX1-107 | 8 | 69 | 11.6 | N.A. | N.A. | — | v |
| EMX1-108 | 5 | 43 | 11.6 | N.A. | N.A. | — | v |
| EMX1-109 | 5 | 43 | 11.6 | N.A. | N.A. | — | v |
| EMX1-110 | 7 | 61 | 11.5 | N.A. | N.A. | — | v |
| EMX1-111 | 7 | 61 | 11.5 | N.A. | N.A. | — | v |
| EMX1-112 | 5 | 44 | 11.4 | 0 | 0 | — | v |
| EMX1-113 | 5 | 44 | 11.4 | 0 | 0 | — | v |
| EMX1-114 | 7 | 62 | 11.3 | 0 | 0 | — | v |
| EMX1-115 | 6 | 53 | 11.3 | 0 | 0 | — | v |
| EMX1-116 | 8 | 71 | 11.3 | N.A. | N.A. | — | v |
| EMX1-117 | 6 | 53 | 11.3 | N.A. | N.A. | — | v |
| EMX1-118 | 6 | 54 | 11.1 | 0 | 0 | — | v |
| EMX1-119 | 5 | 45 | 11.1 | 0 | 0 | — | v |
| EMX1-120 | 5 | 46 | 10.9 | 0 | 0 | — | v |
| EMX1-121 | 6 | 55 | 10.9 | 0 | 0 | — | v |
| EMX1-122 | 6 | 55 | 10.9 | N.A. | N.A. | — | v |
| EMX1-123 | 8 | 75 | 10.7 | 0 | 0 | — | v |
| EMX1-124 | 6 | 56 | 10.7 | N.A. | N.A. | — | v |
| EMX1-125 | 7 | 66 | 10.6 | N.A. | N.A. | — | v |
| EMX1-126 | 5 | 47 | 10.6 | N.A. | N.A. | — | v |
| EMX1-127 | 5 | 47 | 10.6 | N.A. | N.A. | — | v |
| EMX1-128 | 8 | 76 | 10.5 | N.A. | N.A. | — | v |
| EMX1-129 | 5 | 48 | 10.4 | 0 | 0 | — | v |
| EMX1-130 | 5 | 48 | 10.4 | 0 | 0 | — | v |
| EMX1-131 | 5 | 48 | 10.4 | 0 | 0 | — | v |
| EMX1-132 | 5 | 48 | 10.4 | 1 | 0 | — | v |
| EMX1-133 | 5 | 48 | 10.4 | N.A. | N.A. | — | v |
| EMX1-134 | 7 | 68 | 10.3 | 0 | 0 | — | v |
| EMX1-135 | 6 | 59 | 10.2 | N.A. | N.A. | — | v |
| EMX1-136 | 5 | 49 | 10.2 | N.A. | N.A. | — | v |
| EMX1-137 | 7 | 69 | 10.1 | N.A. | N.A. | — | v |
| EMX1-138 | 5 | 50 | 10.0 | 0 | 0 | — | v |
| EMX1-139 | 5 | 50 | 10.0 | 0 | 0 | — | v |
| EMX1-140 | 7 | 44 | 15.9 | 0 | 0 | — | v |
| EMX1-141 | 5 | 40 | 12.5 | 1 | 0 | — | v |
| EMX1-142 | 6 | 49 | 12.2 | 1 | 0 | — | v |

(N.A.: not applicable because there are no cytosines to be deaminated at these sites)

As can be seen in Table 2, the WGS data obtained using the BE-3-treated genomic DNA and intact (BE-3 untreated) genomic DNA showed the observation of C→T conversion at 16 sites (BE-3 treated) and 1 site (BE-3 untreated) among 142 sites of Group B. Of these sites, 70 sites do not contain cytosine at positions 4 to 8, which is a window of BE3-mediated deamination (numbered 1 to 20 in the 5′ to 3′ direction (expressed as N. A. in Table 2).

In order to assess the off-target effect in parts of Groups A and B sites identified Digenome-seq, DNA from HEK293T cells was subjected to targeted deep sequencing and measured for BE3-induced base editing frequencies and Cas9-induced indel frequencies. The results are given in Table 3, below:

TABLE 3

| | Validation by NGS | | | | | |
|---|---|---|---|---|---|---|
| | Indel | | | Base editing | | |
| | frequency (%) | | | frequency (%) | | |
| | (−) | (+) | | (−) | (+) | |
| | Cas9 | Cas9 | Validation | BE3 | BE3 | Validation |
| EMX1-001 | 0.15 | 61.59 | Validated | 0.10 | 49.33 | Validated |
| (on-target) | | | | | | |
| EMX1-002 | 0.01 | 0.01 | Invalidated | 0.16 | 1.05 | Validated |
| EMX1-003 | 0.00 | 7.94 | Validated | 0.24 | 4.04 | Validated |
| EMX1-004 | 0.00 | 0.01 | Validated | 0.16 | 0.93 | Validated |
| EMX1-005 | 0.00 | 8.63 | Validated | 0.05 | 2.47 | Validated |
| EMX1-006 | 0.29 | 38.25 | Validated | 0.04 | 15.59 | Validated |
| EMX1-007 | 0.01 | 0.01 | Invalidated | 0.08 | 0.13 | Validated |
| EMX1-008 | 0.02 | 0.17 | Validated | 0.03 | 0.62 | Validated |
| EMX1-009 | 0.10 | 3.45 | Validated | 0.02 | 0.15 | Validated |
| EMX1-010 | 0.08 | 0.08 | Invalidated | 0.07 | 0.70 | Validated |
| EMX1-034 | 0.00 | 0.00 | Invalidated | 0.33 | 0.40 | Invalidated |
| EMX1-035 | 0.46 | 0.89 | Validated | 0.23 | 0.48 | Validated |
| EMX1-036 | 0.01 | 0.02 | Invalidated | 0.09 | 0.31 | Validated |
| EMX1-037 | 0.01 | 0.23 | Validated | 0.20 | 0.23 | Validated |
| EMX1-038 | 0.01 | 0.01 | Invalidated | 0.14 | 0.16 | Validated |
| EMX1-140 | 0.01 | 0.00 | Invalidated | 0.38 | 0.36 | Invalidated |
| EMX1-141 | 0.00 | 0.00 | Invalidated | 0.30 | 0.37 | Invalidated |
| EMX1-142 | 0.01 | 0.01 | Invalidated | 0.19 | 0.17 | Invalidated |

As is understood from data of Table 3, a total of 18 sites were analyzed and BE3-induced point mutations were observed at 14 sites including the EMX1 on-target sites, with frequencies above noise levels caused by sequencing errors (0.002-0.38%) (at a validation rate of 78%). It is possible that BE3 can induce mutagenesis at the other BE3-associated, Digenome-captured sites with frequencies below background noise levels. Notably, the method is able to identify BE3 off-target sites at which base editing was detected with a frequency of 0.13% or less, demonstrating that Digenome-seq is a highly sensitive method. EMX1-specific Cas9 nucleases induced indels at 9 of 18 sites with frequencies above noise levels, indicating that BE3 and Cas9 off-target sites are often different from each other. Taken together, such results suggest that BE3 off-target sites can be identified using the Digenome-seq data.

As described above, it will be understood by a person having ordinary skill in the technical field to which the present disclosure pertains that the present disclosure may be embodied in other specific forms without departing from the technical spirit or essential characteristics thereof. In this regard, it should be understood that the above-described embodiments are intended to illustrate in every aspect, but are not intended to be limiting. The scope of the invention should be construed to cover all modifications and variations that come within the meaning and range, as well as equivalent concepts thereof, as defined by the appended claims rather than the foregoing description.

What is claimed is:

1. A method for identifying an off-target site of a cytidine deaminase, the method comprising:
(i) introducing into a cell or contacting with DNA separated from a cell (a) a cytidine deaminase or a gene encoding a cytidine deaminase, (b) an inactivated target-specific endonuclease or a gene encoding an inactivated target-specific endonuclease, and (3) a guide RNA or a gene encoding a guide RNA, to induce a single-strand break in the DNA; and
(ii) analyzing a nucleic acid sequence of the single-strand cleaved DNA fragment; and
(iii) identifying a single-strand break site from the nucleic acid sequence data obtained by the analysis.
wherein the inactivated target-specific endonuclease is a Cas9 protein or Cpf1 protein lacking endonuclease activity of cleaving a DNA duplex.

2. The method of claim 1, wherein the inactivated target-specific endonuclease is a Streptococcus pyogenes-derived Cas9 protein which is different in amino acid residue from the wild-type protein at the following positions:
(1) D10, H840, or D10 and H840;
(2) at least one selected from the group consisting of D1135, R1335, and T1337; or
(3) both of the positions of (1) and (2).

3. The method of claim 1, wherein the guide RNA is a double-stranded or single-stranded guide RNA (sgRNA) including CRISPR RNA (crRNA) and trans-activating crRNA (tracrRNA).

4. The method of claim 1, wherein, the cytidine deaminase is APOBEC (apolipoprotein B mRNA editing enzyme, catalytic polypeptide-like), AID (activation-induced cytidine deaminase), CDA (cytidine deaminase), or a combination thereof.

5. The method of claim 1, wherein the cytidine deaminase and the inactivated target-specific endonuclease are used
in a fusion protein form,
in a mixture of the cytidine deaminase or an mRNA coding therefor and the inactivated target-specific endonuclease or an mRNA coding therefore, or
in a form of a plasmid carrying both a cytidine deaminase-encoding gene and an inactivated target-specific endonuclease-encoding gene or plasmids carrying a cytidine deaminase-encoding gene and an inactivated target-specific endonuclease-encoding gene, respectively.

6. The method of claim 1, wherein the method does not use a uracil-specific excision reagent (USER), which is a uracil DNA glycosylase (DG), endonuclease VIII, or a combination thereof.

7. The method of claim 1, being carried out in vitro.

8. The method of claim 1, wherein the DNA separated from cells are a genomic DNA.

9. The method of claim 1, wherein the DNA separated from cells in step (i) is a genomic DNA and the nucleic acid sequence analysis of step (iii) is carried out by whole genome sequencing.

10. The method of claim 1, further comprising a step of determining the cleaved site as an off-target site when the cleaved site is not an on-target site, after step (iv).

11. The method of claim 10, wherein the step (iii) includes a step of identifying a number of DNA reads of which the 5′ ends are vertically aligned.

12. The method of claim 11, wherein the off-target site has two or more DNA reads of which the 5′ ends are vertically aligned.

13. The method of claim 12, wherein the off-target site corresponds to at least one of the following conditions:
a strand complementary to the strand on which a break has been induced in a DNA fragment includes a PAM sequence;
a complementary strand to the strand on which a break has been induced in a DNA fragment includes 15 or fewer nucleotide mismatches with a sequence at the on-target site; and
a complementary strand to the strand on which a break has been induced in a DNA fragment includes conversion of at least cytosine (C) residue to uracil (U) or thymine (T).

14. A method for analyzing a nucleic acid sequence of DNA to which base editing is introduced by a cytidine deaminase, the method comprising the steps of:
(i) introducing into a cell or contacting with DNA separated from a cell (a) a cytidine deaminase or a gene encoding a cytidine deaminase, (b) an inactivated target-specific endonuclease or a gene encoding an inactivated target-specific endonuclease, and (3) a guide RNA or a gene encoding a guide RNA, to induce a single-strand break in the DNA; and
(ii) analyzing a nucleic acid sequence of a DNA fragment in which the single-strand break has been induced,
wherein the inactivated target-specific endonuclease is a Cas9 protein or Cpf1 protein lacking the endonuclease activity of cleaving a DNA duplex.

15. The method of claim 14, wherein the inactivated target-specific endonuclease is a Streptococcus pyogenes-derived Cas9 protein which is different in amino acid residue from the wild-type protein at the following positions:
(1) D10, H840, or D10 and H840;
(2) at least one selected from the group consisting of D1135, R1335, and T1337; or
(3) both of the positions of (1) and (2).

16. The method of claim 14, wherein the cytidine deaminase is APOBEC (apolipoprotein B mRNA editing enzyme, catalytic polypeptide-like), AID (activation-induced cytidine deaminase), CDA (cytidine deaminase), or a combination thereof.

17. The method of claim 14, wherein the method does not use a uracil-specific excision reagent (USER), which is a uracil DNA glycosylase (DG), endonuclease VIII, or a combination thereof.

18. Another aspect provides a method for identifying a base-editing site of a cytidine deaminase, the method comprising the steps of:
(i) introducing to a cell or contacting with DNA separated from a cell (a) a cytidine deaminase or a gene encoding a cytidine deaminase, (b) an inactivated target-specific endonuclease or a gene encoding an inactivated target-specific endonuclease, and (3) a guide RNA or a gene encoding a guide RNA, to induce a single-strand break in DNA;
(ii) analyzing a nucleic acid sequence of the DNA fragment in which the single-strand break has been induced; and
(iii) identifying a single-strand break site from the sequence reads obtained by the analysis,
wherein the inactivated target-specific endonuclease is a Cas9 protein or Cpfl protein lacking the endonuclease activity of cleaving a DNA duplex.

19. The method of claim 18, wherein the inactivated target-specific endonuclease is a Streptococcus pyogenes-derived Cas9 protein which is different in amino acid residue from the wild-type protein at the following positions:
(1) D10, H840, or D10 and H840;
(2) at least one selected from the group consisting of D1135, R1335, and T1337; or
(3) both of the positions of (1) and (2).

20. The method of claim 18, wherein the method does not use a uracil-specific excision reagent (USER), which is a uracil DNA glycosylase (DG), endonuclease VIII, or a combination thereof.


SEQUENCE LISTING (159 sequences)

SEQ ID NO: 1 - RNA, 12 nt, Artificial Sequence
  Essential part of crRNA
guuuuagagcua

SEQ ID NO: 2 - RNA, 10 nt, Artificial Sequence
  3′-terminal part of crRNA
ugcuguuuug

SEQ ID NO: 3 - RNA, 60 nt, Artificial Sequence
  Essential part of tracrRNA
uagcaaguuaaaauaaggcuaguccguuaucaacuugaaaaaguggcaccgagucggugc

SEQ ID NO: 4 - protein, 1368 aa, Artificial Sequence [sequence omitted]

SEQ ID NO: 5 - DNA, 4107 bp, Artificial Sequence
  Cas9-coding sequence
atggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactaa

SEQ ID NO: 6 - protein, 229 aa, Artificial Sequence [sequence omitted]

SEQ ID NO: 7 - DNA, 5148 bp, Artificial Sequence
  BE3 coding sequence
catcatcatcatcatcacatgtcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgttcatctacatcgcgcgtctgtaccaccacgcggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgaccttcttcaccatcgcgctgcagtcttgccactaccagcgtctgccgccgcacatcctgtgggcgaccggtctgaaatccggtagcgaaacaccggggacttcagaatcggccaccccggagtctgataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtc

SEQ ID NO: 8 - DNA, 18 bp, Artificial Sequence
  Hisx6
catcatcatcatcatcac

SEQ ID NO: 9 - DNA, 687 bp, Artificial Sequence
  rAPOBEC1
atgtcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgttcatctacatcgcgcgtctgtaccaccacgcggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgaccttcttcaccatcgcgctgcagtcttgccactaccagcgtctgccgccgcacatcctgtgggcgaccggtctgaaa

SEQ ID NO: 10 - DNA, 48 bp, Artificial Sequence
  XTEN (linker)
tccggtagcgaaacaccggggacttcagaatcggccaccccggagtct

SEQ ID NO: 11 - DNA, 4101 bp, Artificial Sequence
  coding gene of Cas9 (D10A, nickase)
gataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 12 - DNA, 21 bp, Artificial Sequence
  NLS
cccaagaagaagaggaaagtc

SEQ ID NO: 13 - DNA, 12 bp, Artificial Sequence
  linker
tctggtggttct

SEQ ID NO: 14 - DNA, 23 bp, Artificial Sequence
  EMX1 on-target sequence
gagtccgagcagaagaagaaggg

SEQ ID NO: 15 - DNA, 18 bp, Artificial Sequence
  1st PCR forward primer for targeted deep sequencing
agtgttgaggccccagtg

SEQ ID NO: 16 - DNA, 53 bp, Artificial Sequence
  1st PCR reverse primer for targeted deep sequencing
gtgactggagttcagacgtgtgctcttccgatctcagcagcaagcagcactct

SEQ ID NO: 17 - DNA, 53 bp, Artificial Sequence
  2nd PCR forward primer for targeted deep sequencing
acactctttccctacacgacgctcttccgatctgggcctcctgagtttctcat

SEQ ID NO: 18 - DNA, 53 bp, Artificial Sequence; identical to SEQ ID NO: 16
  2nd PCR reverse primer for targeted deep sequencing

SEQ ID NO: 19 - DNA, 22 bp, Artificial Sequence
  EMX1-002 off-target sequence
gaatccaagagaagaagaatgg

SEQ ID NO: 20 - DNA, 23 bp, Artificial Sequence
  EMX1-003 off-target sequence
gaggccgagcagaagaaagacgg

SEQ ID NO: 21 - DNA, 23 bp, Artificial Sequence
  EMX1-004 off-target sequence
gaatccaagcagaagaagagaag

SEQ ID NO: 22 - DNA, 23 bp, Artificial Sequence
  EMX1-005 off-target sequence
gagtcctagcaggagaagaagag

SEQ ID NO: 23 - DNA, 23 bp, Artificial Sequence
  EMX1-006 off-target sequence
gagtctaagcagaagaagaagag

SEQ ID NO: 24 - DNA, 23 bp, Artificial Sequence
  EMX1-007 off-target sequence
gagtccaagcagtagaggaaggg

SEQ ID NO: 25 - DNA, 23 bp, Artificial Sequence
  EMX1-008 off-target sequence
acgtctgagcagaagaagaatgg

SEQ ID NO: 26 - DNA, 23 bp, Artificial Sequence
  EMX1-009 off-target sequence
aagtctgagcacaagaagaatgg

SEQ ID NO: 27 - DNA, 22 bp, Artificial Sequence
  EMX1-010 off-target sequence
gtgtcctagagaagaagaaggg

SEQ ID NO: 28 - DNA, 23 bp, Artificial Sequence
  EMX1-011 off-target sequence
gagttagagcagaagaagaaagg

SEQ ID NO: 29 - DNA, 22 bp, Artificial Sequence
  EMX1-012 off-target sequence
gagacagagagaagaagaatgg

SEQ ID NO: 30 - DNA, 23 bp, Artificial Sequence
  EMX1-013 off-target sequence
gagcctgagcggaagaggaaagg

SEQ ID NO: 31 - DNA, 23 bp, Artificial Sequence
  EMX1-014 off-target sequence
aatacagagcagaagaagaatgg

SEQ ID NO: 32 - DNA, 23 bp, Artificial Sequence
  EMX1-015 off-target sequence
agtccagagcaaaataagaaggg

SEQ ID NO: 33 - DNA, 23 bp, Artificial Sequence
  EMX1-016 off-target sequence
gagcctgagcagaaggagaaggg

SEQ ID NO: 34 - DNA, 23 bp, Artificial Sequence
  EMX1-017 off-target sequence
gaagaagagcaaaagaagaaggg

SEQ ID NO: 35 - DNA, 23 bp, Artificial Sequence
  EMX1-018 off-target sequence
gtgacagagcaaaagaagaaagg

SEQ ID NO: 36 - DNA, 23 bp, Artificial Sequence
  EMX1-019 off-target sequence
gaagaggagcaaaagaagaaggg

SEQ ID NO: 37 - DNA, 23 bp, Artificial Sequence
  EMX1-020 off-target sequence
aagtccagacagaagaagaagga

SEQ ID NO: 38 - DNA, 23 bp, Artificial Sequence
  EMX1-021 off-target sequence
aaatccaaccagaagaagaaagg

SEQ ID NO: 39 - DNA, 23 bp, Artificial Sequence
  EMX1-022 off-target sequence
gttcaagagcaggagaagaaggg

SEQ ID NO: 40 - DNA, 23 bp, Artificial Sequence
  EMX1-023 off-target sequence
gagtaagagaagaagaagaaggg

SEQ ID NO: 41 - DNA, 23 bp, Artificial Sequence
  EMX1-024 off-target sequence
aagcccgagcaaaggaagaaagg

SEQ ID NO: 42 - DNA, 23 bp, Artificial Sequence
  EMX1-025 off-target sequence
cctatagagcaaaagaagaaagg

SEQ ID NO: 43 - DNA, 23 bp, Artificial Sequence
  EMX1-026 off-target sequence
gatcaagagaagaagaagaaggg

SEQ ID NO: 44 - DNA, 23 bp, Artificial Sequence
  EMX1-027 off-target sequence
caaaaagagcaaaagaagaacgg

SEQ ID NO: 45 - DNA, 23 bp, Artificial Sequence
  EMX1-028 off-target sequence
tacagtgagcaaaagaagaaggg

SEQ ID NO: 46 - DNA, 23 bp, Artificial Sequence
  EMX1-029 off-target sequence
gttcaagagcaggagaagaaggg

SEQ ID NO: 47 - DNA, 23 bp, Artificial Sequence
  EMX1-030 off-target sequence
ttctcagagcaaaagaagaatgg

SEQ ID NO: 48 - DNA, 23 bp, Artificial Sequence
  EMX1-031 off-target sequence
ctttgccaggagaaggacattgc

SEQ ID NO: 49 - DNA, 23 bp, Artificial Sequence
  EMX1-032 off-target sequence
aggttagagcaaaagaagaaagg

SEQ ID NO: 50 - DNA, 23 bp, Artificial Sequence
  EMX1-033 off-target sequence
tatacggagcagaagaagaatgg

SEQ ID NO: 51 - DNA, 23 bp, Artificial Sequence
  EMX1-034 off-target sequence
gagtccacacagaagaagaaaga

SEQ ID NO: 52 - DNA, 23 bp, Artificial Sequence
  EMX1-035 off-target sequence
gaatccaagcaggagaagaagga

SEQ ID NO: 53 - DNA, 23 bp, Artificial Sequence
  EMX1-036 off-target sequence
aagtccatgcagaagaggaaggg

SEQ ID NO: 54 - DNA, 23 bp, Artificial Sequence
  EMX1-037 off-target sequence
aagtccgaggagaggaagaaagg

SEQ ID NO: 55 - DNA, 22 bp, Artificial Sequence
  EMX1-038 off-target sequence
aagtccaagtgaagaagaaagg

SEQ ID NO: 56 - DNA, 23 bp, Artificial Sequence
  EMX1-039 off-target sequence
gagaacgagaagaaagaggagag

SEQ ID NO: 57 - DNA, 23 bp, Artificial Sequence
  EMX1-040 off-target sequence
gagagagagagagagagggaggg

SEQ ID NO: 58 - DNA, 23 bp, Artificial Sequence
  EMX1-041 off-target sequence
ctggcagagcaaaagaagagggg

SEQ ID NO: 59 - DNA, 23 bp, Artificial Sequence
  EMX1-042 off-target sequence
tcatccaagcagaagaagaagag

SEQ ID NO: 60 - DNA, 23 bp, Artificial Sequence
  EMX1-043 off-target sequence
gtacaggagcaggagaagaatgg

SEQ ID NO: 61 - DNA, 23 bp, Artificial Sequence
  EMX1-044 off-target sequence
aagaaggagaagaagaagaaggg

SEQ ID NO: 62 - DNA, 23 bp, Artificial Sequence
  EMX1-045 off-target sequence
gagagagagagagagagaaaggg

SEQ ID NO: 63 - DNA, 23 bp, Artificial Sequence
  EMX1-046 off-target sequence
gagccggagcagaagaaggaggg

SEQ ID NO: 64 - DNA, 23 bp, Artificial Sequence
  EMX1-047 off-target sequence
gagggagagagggagagagaaag

SEQ ID NO: 65 - DNA, 23 bp, Artificial Sequence
  EMX1-048 off-target sequence
ttatctccgagaaggaagaaggg

SEQ ID NO: 66 - DNA, 23 bp, Artificial Sequence
  EMX1-049 off-target sequence
gattctgtcccgaatcagaaggg

SEQ ID NO: 67 - DNA, 23 bp, Artificial Sequence
  EMX1-050 off-target sequence
atgcaagagaagaagaagaaagg

SEQ ID NO: 68 - DNA, 23 bp, Artificial Sequence
  EMX1-051 off-target sequence
agcagggagcagaggaagaatgg

SEQ ID NO: 69 - DNA, 23 bp, Artificial Sequence
  EMX1-052 off-target sequence
gagaaggagaagaagaagaaggg

SEQ ID NO: 70 - DNA, 23 bp, Artificial Sequence
  EMX1-053 off-target sequence
actctagagcagaaaaagaatgg

SEQ ID NO: 71 - DNA, 23 bp, Artificial Sequence
  EMX1-054 off-target sequence
gaggaggaggaggaggaaggagg

SEQ ID NO: 72 - DNA, 23 bp, Artificial Sequence
  EMX1-055 off-target sequence
aaagaggagaagaagaagaaggg

SEQ ID NO: 73 - DNA, 23 bp, Artificial Sequence
  EMX1-056 off-target sequence
ggggaggagcagaagaagagagg

SEQ ID NO: 74 - DNA, 23 bp, Artificial Sequence
  EMX1-057 off-target sequence
aggccgaggcaggagaataggag

SEQ ID NO: 75 - DNA, 23 bp, Artificial Sequence
  EMX1-058 off-target sequence
gagaaggagcagaaaaagaatgg

SEQ ID NO: 76 - DNA, 23 bp, Artificial Sequence
  EMX1-059 off-target sequence
acagtagagcagaagaagactgg

SEQ ID NO: 77 - DNA, 23 bp, Artificial Sequence
  EMX1-060 off-target sequence
atccaagagcaggagaagaaggg

SEQ ID NO: 78 - DNA, 23 bp, Artificial Sequence
  EMX1-061 off-target sequence
ataagagagcaaaagaagaaagg

SEQ ID NO: 79 - DNA, 23 bp, Artificial Sequence
  EMX1-062 off-target sequence
gagagagagagagagagaaacgg

SEQ ID NO: 80 - DNA, 23 bp, Artificial Sequence
  EMX1-063 off-target sequence
gagaaggagaagaaggagaagag

SEQ ID NO: 81 - DNA, 23 bp, Artificial Sequence
  EMX1-064 off-target sequence
aagaagaagaagaagaagaagag

SEQ ID NO: 82 - DNA, 23 bp, Artificial Sequence
  EMX1-065 off-target sequence
tttcgggagcagaagaagaacag

SEQ ID NO: 83 - DNA, 23 bp, Artificial Sequence
  EMX1-066 off-target sequence
atcacagagcaggagaagaaggg

SEQ ID NO: 84 - DNA, 23 bp, Artificial Sequence
  EMX1-067 off-target sequence
aaacaggagcagaggaagaaggg

SEQ ID NO: 85 - DNA, 23 bp, Artificial Sequence
  EMX1-068 off-target sequence
aagaagaggaagaggaggaaaag

SEQ ID NO: 86 - DNA, 23 bp, Artificial Sequence
  EMX1-069 off-target sequence
gaaatggagaagaagaagaaggg

SEQ ID NO: 87 - DNA, 23 bp, Artificial Sequence
  EMX1-070 off-target sequence
gagagagaaagaaagaaaaagga

SEQ ID NO: 88 - DNA, 23 bp, Artificial Sequence
  EMX1-071 off-target sequence
gctgtggagcaaaagaagaaagg

SEQ ID NO: 89 - DNA, 23 bp, Artificial Sequence
  EMX1-072 off-target sequence
gaggaggagcagaagaagaaaag

SEQ ID NO: 90 - DNA, 23 bp, Artificial Sequence
  EMX1-073 off-target sequence
taaaaggagcagaaaaagaaggg

SEQ ID NO: 91 - DNA, 23 bp, Artificial Sequence
  EMX1-074 off-target sequence
taccttgagcaaaagaagaaggg

SEQ ID NO: 92 - DNA, 23 bp, Artificial Sequence
  EMX1-075 off-target sequence
aagaaggagaagaagaagaaggg

SEQ ID NO: 93 - DNA, 23 bp, Artificial Sequence
  EMX1-076 off-target sequence
gcaggagagcagaagaagaaagg

SEQ ID NO: 94 - DNA, 23 bp, Artificial Sequence
  EMX1-077 off-target sequence
aagagcgagattaagaggaatgg

SEQ ID NO: 95 - DNA, 23 bp, Artificial Sequence
  EMX1-078 off-target sequence
aagaaaggagaggagaggagagg

SEQ ID NO: 96 - DNA, 23 bp, Artificial Sequence
  EMX1-079 off-target sequence
agtagtgagcagaagaagatagg

SEQ ID NO: 97 - DNA, 23 bp, Artificial Sequence
  EMX1-080 off-target sequence
aagaaggagaagaagaagaagag

SEQ ID NO: 98 - DNA, 23 bp, Artificial Sequence
  EMX1-081 off-target sequence
gagagagagagagagagaaagag

SEQ ID NO: 99 - DNA, 23 bp, Artificial Sequence
  EMX1-082 off-target sequence
ggccaggagcaggagaagaaggg

SEQ ID NO: 100 - DNA, 23 bp, Artificial Sequence
  EMX1-083 off-target sequence
agaggagagcagaagaaggatgg

SEQ ID NO: 101 - DNA, 23 bp, Artificial Sequence
  EMX1-084 off-target sequence
aagaaggagaagaagaaggagga

SEQ ID NO: 102 - DNA, 23 bp, Artificial Sequence
  EMX1-085 off-target sequence
cattaagagcaggagaagaaggg

SEQ ID NO: 103 - DNA, 23 bp, Artificial Sequence
  EMX1-086 off-target sequence
cgctgatacagaaagaagaatgg

SEQ ID NO: 104 - DNA, 23 bp, Artificial Sequence
  EMX1-087 off-target sequence
gaagtggagcaggagaagaaggg

SEQ ID NO: 105 - DNA, 23 bp, Artificial Sequence
  EMX1-088 off-target sequence
tttgtggagcagaaaaagaaagg

SEQ ID NO: 106 - DNA, 23 bp, Artificial Sequence
  EMX1-089 off-target sequence
aagtcagaggagaagaagaaggg

SEQ ID NO: 107 - DNA, 23 bp, Artificial Sequence
  EMX1-090 off-target sequence
aaagctgagcagaaaaagaaggg

SEQ ID NO: 108 - DNA, 23 bp, Artificial Sequence
  EMX1-091 off-target sequence
gcagtggagcagaagaagatggg

SEQ ID NO: 109 - DNA, 23 bp, Artificial Sequence
  EMX1-092 off-target sequence
acaaaagagcagaggaagaaagg

SEQ ID NO: 110 - DNA, 23 bp, Artificial Sequence
  EMX1-093 off-target sequence
gaagtagagcagaagaagaagcg

SEQ ID NO: 111 - DNA, 23 bp, Artificial Sequence
  EMX1-094 off-target sequence
aaagagggagagaagaagaaggg

SEQ ID NO: 112 - DNA, 23 bp, Artificial Sequence
  EMX1-095 off-target sequence
aagaaggagaagaagaagaagag

SEQ ID NO: 113 - DNA, 23 bp, Artificial Sequence
  EMX1-096 off-target sequence
aaggccaagcagaagagtaatgg

SEQ ID NO: 114 - DNA, 23 bp, Artificial Sequence
  EMX1-097 off-target sequence
acacaagagcagaagaagaaaga

SEQ ID NO: 115 - DNA, 23 bp, Artificial Sequence
  EMX1-098 off-target sequence
gccaatgagcaggagaagaaggg

SEQ ID NO: 116 - DNA, 23 bp, Artificial Sequence
  EMX1-099 off-target sequence
catatagagcaaaagaagagagg

SEQ ID NO: 117 - DNA, 23 bp, Artificial Sequence
  EMX1-100 off-target sequence
gaggtggaggggaagagggaagg

SEQ ID NO: 118 - DNA, 23 bp, Artificial Sequence
  EMX1-101 off-target sequence
gagagagagagagagagaaaggg

SEQ ID NO: 119 - DNA, 23 bp, Artificial Sequence
  EMX1-102 off-target sequence
aaggaggagaagaagaagaaggg

SEQ ID NO: 120 - DNA, 23 bp, Artificial Sequence
  EMX1-103 off-target sequence
gagggagagagagagagagaaag

SEQ ID NO: 121 - DNA, 23 bp, Artificial Sequence
  EMX1-104 off-target sequence
agtcgggagcaggagaagaaagg

SEQ ID NO: 122 - DNA, 23 bp, Artificial Sequence
  EMX1-105 off-target sequence
gttcaagagcagaagaagaatgg

SEQ ID NO: 123 - DNA, 23 bp, Artificial Sequence
  EMX1-106 off-target sequence
tctaaagagcagaaaaagaaagg

SEQ ID NO: 124 - DNA, 23 bp, Artificial Sequence
  EMX1-107 off-target sequence
actgatgagcagaagaagaaagg

SEQ ID NO: 125 - DNA, 23 bp, Artificial Sequence
  EMX1-108 off-target sequence
aagaaggagaagaagaaggagga

SEQ ID NO: 126 - DNA, 23 bp, Artificial Sequence
  EMX1-109 off-target sequence
aggaaaagagagaagaagaaggg

SEQ ID NO: 127 - DNA, 23 bp, Artificial Sequence
  EMX1-110 off-target sequence
gaggaggagaagaagaagaaggg

SEQ ID NO: 128 - DNA, 23 bp, Artificial Sequence
  EMX1-111 off-target sequence
gagagagagagagagagaaaggg

SEQ ID NO: 129 - DNA, 23 bp, Artificial Sequence
  EMX1-112 off-target sequence
gaggccgaagctgaggtgggagg

SEQ ID NO: 130 - DNA, 23 bp, Artificial Sequence
  EMX1-113 off-target sequence
gagcccaagaagaagaagaagga

SEQ ID NO: 131 - DNA, 23 bp, Artificial Sequence
  EMX1-114 off-target sequence
atgtacagagagaagaagaaagg

SEQ ID NO: 132 - DNA, 23 bp, Artificial Sequence
  EMX1-115 off-target sequence
gccttcagagagaagaagaatgg

SEQ ID NO: 133 - DNA, 23 bp, Artificial Sequence
  EMX1-116 off-target sequence
ggagtagagcagaggaagaaggg

SEQ ID NO: 134 - DNA, 23 bp, Artificial Sequence
  EMX1-117 off-target sequence
gaagtagagcagaagaagaagcg

SEQ ID NO: 135 - DNA, 23 bp, Artificial Sequence
  EMX1-118 off-target sequence
gctcctgagcagaagaagaacag

SEQ ID NO: 136 - DNA, 23 bp, Artificial Sequence
  EMX1-119 off-target sequence
agttcagagcaggagaagaatgg

SEQ ID NO: 137 - DNA, 23 bp, Artificial Sequence
  EMX1-120 off-target sequence
atgaacagagagaagaagaatgg

SEQ ID NO: 138 - DNA, 23 bp, Artificial Sequence
  EMX1-121 off-target sequence
gagagcgagagagagagagaggg

SEQ ID NO: 139 - DNA, 23 bp, Artificial Sequence
  EMX1-122 off-target sequence
aaaaaggagcagaagaagaagga

SEQ ID NO: 140 - DNA, 23 bp, Artificial Sequence
  EMX1-123 off-target sequence
ttctccaagcagaagaagaagag

SEQ ID NO: 141 - DNA, 23 bp, Artificial Sequence
  EMX1-124 off-target sequence
gagagagagagagagagaaaggg

SEQ ID NO: 142 - DNA, 23 bp, Artificial Sequence
  EMX1-125 off-target sequence
ttggaaagagagaagaagaaggg

SEQ ID NO: 143 - DNA, 23 bp, Artificial Sequence
  EMX1-126 off-target sequence
aagaggaagaagaagaagaagag

SEQ ID NO: 144 - DNA, 23 bp, Artificial Sequence
  EMX1-127 off-target sequence
gagagagagaaagagaaagagag

SEQ ID NO: 145 - DNA, 23 bp, Artificial Sequence
  EMX1-128 off-target sequence
gaagaagagaagaagaagaaggg

SEQ ID NO: 146 - DNA, 23 bp, Artificial Sequence
  EMX1-129 off-target sequence
gattctgagaaaattaagatggg

SEQ ID NO: 147 - DNA, 23 bp, Artificial Sequence
  EMX1-130 off-target sequence
gggctccggcagaagatgccatg

SEQ ID NO: 148 - DNA, 23 bp, Artificial Sequence
  EMX1-131 off-target sequence
gattccaagcaatggaggagggg

SEQ ID NO: 149 - DNA, 23 bp, Artificial Sequence
  EMX1-132 off-target sequence
gtccaagagcaggagaagaaggg

SEQ ID NO: 150 - DNA, 23 bp, Artificial Sequence
  EMX1-133 off-target sequence
atctgggagcaggagaagaaggg

SEQ ID NO: 151 - DNA, 23 bp, Artificial Sequence
  EMX1-134 off-target sequence
gaggatccgaggatgtagaaggg

SEQ ID NO: 152 - DNA, 23 bp, Artificial Sequence
  EMX1-135 off-target sequence
gaagaagaagaaagaaagaaaga

SEQ ID NO: 153 - DNA, 23 bp, Artificial Sequence
  EMX1-136 off-target sequence
cagagagagaataagtagcatag

SEQ ID NO: 154 - DNA, 23 bp, Artificial Sequence
  EMX1-137 off-target sequence
tgaggaagagagaagaagaaagg

SEQ ID NO: 155 - DNA, 23 bp, Artificial Sequence
  EMX1-138 off-target sequence
caagcatgagagaagaagatggg

SEQ ID NO: 156 - DNA, 23 bp, Artificial Sequence
  EMX1-139 off-target sequence
gagcacgagcaagagaagaaggg

SEQ ID NO: 157 - DNA, 23 bp, Artificial Sequence
  EMX1-140 off-target sequence
gagtcccagcaaaagaagaaaag

SEQ ID NO: 158 - DNA, 22 bp, Artificial Sequence
  EMX1-141 off-target sequence
gagtccctcaggagaagaaagg

SEQ ID NO: 159 - DNA, 22 bp, Artificial Sequence
  EMX1-142 off-target sequence
gagtccttgagaagaaggaagg